(19) United States
(12) Patent Application Publication
(10) Pub. No.: US 2017/0196828 A1
(43) Pub. Date: Jul. 13, 2017

(54) Continuous Administration of Levodopa and/or Dopa Decarboxylase Inhibitors and Compositions for Same

(71) Applicant: NeuroDerm, Ltd., Rehovot (IL)

(72) Inventors: Oron Yacoby-Zeevi, Moshav Bitsaron (IL); Mara Nemas, Gedera (IL)

(21) Appl. No.: 15/244,326

(22) Filed: Aug. 23, 2016

Related U.S. Application Data
(63) Continuation of application No. 13/796,232, filed on Mar. 12, 2013, now abandoned.
Application No. 13/796,232 is a continuation-in-part of application No. 12/961,534, filed on Dec. 7, 2010, now Pat. No. 9,101,663.
Application No. 12/961,534 is a continuation of application No. 12/836,130, filed on Jul. 14, 2010, now Pat. No. 7,863,336.
Application No. 12/836,130 is a continuation of application No. 12/781,357, filed on May 17, 2010, now Pat. No. 8,193,243.
(60) Provisional application No. 61/179,511, filed on May 19, 2009.

(51) Int. Cl. A61K 31/245; A61K 9/00; A61K 31/655; A61K 9/08

(52) U.S. Cl. CPC A61K 31/245; A61K 9/08; A61K 9/0019; A61K 9/0014; A61K 9/0053; A61K 31/655

(57) ABSTRACT

Disclosed herein are for example, liquid aqueous compositions that include for example an ester or salt of levodopa, or an ester or salt of carbidopa, and methods for treating neurological or movement diseases or disorders such as restless leg syndrome, Parkinson's disease, secondary parkinsonism, Huntington's disease, Parkinson's like syndrome, PSP, MSA, ALS, Shy-Drager syndrome, dystonia, and conditions resulting from brain injury including carbon monoxide or manganese intoxication, using substantially continuous administration of levodopa and/or carbidopa or ester and/or salt thereof.

### RELATED APPLICATIONS

This application is a continuation of U.S. Ser. No. 13/796,232, filed Mar. 12, 2013, which is a continuation-in-part of U.S. Ser. No. 12/961,534, filed Dec. 7, 2010, which is a continuation of U.S. Ser. No. 12/836,130, filed Jul. 14, 2010, which is a continuation of Ser. No. 12/781,357, filed May 17, 2010, which in turn claims priority to U.S. Ser. No. 61/179,511, filed May 19, 2009, each of which is hereby incorporated by reference in its entirety.

### BACKGROUND

Parkinson's disease is a degenerative condition characterized by reduced concentration of the neurotransmitter dopamine in the brain. Levodopa (L-dopa or L-3,4-dihydroxyphenylalanine) is an immediate metabolic precursor of dopamine that, unlike dopamine, is able to cross the blood-brain barrier, and is most commonly used for restoring the dopamine concentration in the brain. For the past 40 years, levodopa has remained the most effective therapy for the treatment of Parkinson's disease.

However, levodopa has a short half-life in plasma that, even under best common current standard of care, results in pulsatile dopaminergic stimulation. Long-term therapy is therefore complicated by motor fluctuations and dyskinesia that can represent a source of significant disability for some patients. A therapeutic strategy that could ultimately deliver levodopa/dopamine to the brain in a more continuous and physiologic manner would provide the benefits of standard levodopa with reduced motor complications and is much needed by patients suffering from Parkinson's disease and other neurological or movement disorders (Olanow C W; Mov. Dis. 2008, 23 (Suppl. 3):S 613-S622). Sustained-release oral levodopa formulations have been developed, but, at best, such preparations have been found to be no more efficacious than standard tablets. Continuous administration of levodopa by intraduodenal administration or infusion has also been attempted by using ambulatory pumps or patches. Such treatments, especially intraduodenal, are extremely invasive and inconvenient. Further, such treatments may be associated with dopaminergic adverse events; continuous administration of levodopa or dopa agonists is still associated with off periods that are self-limiting despite continued delivery of the drug. Nutt J G; Mov. Dis. 2008, 23 (Suppl. 3): S580-4.

The metabolic transformation of levodopa to dopamine is catalyzed by the aromatic L-amino acid decarboxylase enzyme, a ubiquitous enzyme with particularly high concentrations in the intestinal mucosa, liver, brain and brain capillaries. Due to the possibility of extracerebral metabolism of levodopa, it is necessary to administer large doses of levodopa leading to high extracerebral concentrations of dopamine that cause nausea in some patients. Therefore, levodopa is usually administered concurrently with oral administration of a dopa decarboxylase inhibitor, such as carbidopa or benserazide, which reduces by 60-80% the levodopa dose required for a clinical response, and thus prevents certain of its side effects by inhibiting the conversion of levodopa to dopamine outside the brain. Exactly how this dose reduction is accomplished is uncertain. Various formulations comprising levodopa alone or together with inhibitors of enzymes associated with the metabolic degradation of levodopa are well known, for example, decarboxylase inhibitors such as carbidopa and benserazide, catechol-O-methyl transferase (COMT) inhibitors such as entacapone and tolcapone, and monoamine oxidase (MAO)-A or MAO-B inhibitors such as moclobemide, rasagiline or selegiline or safinamide. Currently available oral drugs include SINEMET® and SINEMET® CR sustained-release tablets that include carbidopa or levodopa; STALEVO®tablets containing carbidopa, entacapone and levodopa; and MADOPAR® tablets containing levodopa and benserazide. There is an on-going and urgent need for methods and compositions that can effect continuous stimulation of L-dopa to more effectively treat movement disorders such as Parkinson's disease.

Carbidopa [(-)-L-α-hydrazine-α-methyl-β-(3,4-dihydroxybenzene) propanoic acid monohydrate], a white, crystalline compound, only slightly soluble in water, is a dopa decarboxylase inhibitor commonly administered with levodopa. Only 40-70% of an oral dose of carbidopa is absorbed in man, monkey and dog. Although carbidopa has been orally administered with levodopa for over 30 years, no stable liquid formulation having e.g., an effective concentration in a volume suitable for use for subcutaneous or transdermal delivery has ever been achieved. There is an urgent, long standing need for such carbidopa formulations that can be administered more easily to patients, especially as compared to current invasive modes such as duodenal administration.

### SUMMARY

This disclosure relates at least in part to the discovery that an ester or salt of levodopa or carbidopa can form a stable, liquid aqueous formulation, suitable for e.g., continuous subcutaneous, transdermal, intradermal, intravenous and/or intraduodenal administration, at a physiologically acceptable pH. Such disclosed compositions are capable of substantially continuously administering levodopa and/or carbidopa to a patient in need thereof. For example, disclosed herein are compositions that relate to the disclosed discovery that are capable of substantially continuously administering levodopa and/or a dopa decarboxylase inhibitor such as carbidopa, optionally together with discrete (e.g. oral) co-administration of levodopa or carbidopa, may stimulate L-dopa substantially continuously and thus e.g., extend the effectiveness of a levodopa oral dosing regimen and/or reduce the daily dosage of levodopa, while effectively treating a movement and/or neurological disorder such as Parkinson's disease.

In an embodiment, disclosed here in a pharmaceutically acceptable liquid composition comprising a levodopa ester or pharmaceutically acceptable salt thereof, wherein the levodopa ester is selected from the group consisting of: levodopa methyl ester, levodopa ethyl ester, levodopa propyl ester, levodopa isopropyl ester and levodopa benzyl ester; and water.

For example, the disclosure provides for an ester of levodopa or carbidopa that is suitable for e.g., continuous subcutaneous, transdermal, intradermal, intravenous, oral, or intraduodenal administration.

Also contemplated herein are liquid (e.g., liquid at room temperature) or gel formulations or compositions that include an ester or salt of levodopa, or ester or salt of carbidopa, e.g., an arginine salt of carbidopa, e.g., include carbidopa and arginine, that may be suitable for substantially continuous administration to a patient e.g. with or without use of, for example, a transdermal patch or subcutaneous pump (e.g. an insulin-like pump). Such contemplated liquid compositions, e.g., that include a disclosed levodopa ester and/or a salt thereof, may include at least 5% (w/v), at least 10% (w/v), at least 20% (w/v), at least 50% (w/v) or more by weight levodopa, (e.g. about 20% to about 60% (w/v) (or about 5% to about 40% (w/v)) levodopa). A liquid composition that includes levodopa, as contemplated herein, may have a physiologically acceptable pH, e.g. a pH of about 4.0 to 9.5, e.g., about 4 to about 6, or about 4 to about 7, at 25° C.

Exemplary liquid compositions contemplated herein may be liquid solutions, e.g. may be a substantially homogenous mixture that includes a disclosed levodopa ester, and may include water. In other embodiments, contemplated compositions may also include other active agents such as carbidopa, entacapone and/or tolcapone and/or salts or esters thereof.

In some embodiments, disclosed compositions, e.g. liquid compositions, may be substantially stable at 25° C. for at least about 48 hours or more. Such stability may result in minimal (e.g. less than 1%, less than 3% or less than 5%) hydrolyzation of levodopa or carbidopa (e.g., esters and/or salts thereof).

In one aspect, provided herein is a kit comprising a first formulation suitable for continuous administration to a patient comprising a disclosed carbidopa salt, carbidoba ester or carbidoba ester salt formation with or without a disclosed levodopa salt, levodopa ester or levodopa ester salt formulation, and a second formulation suitable for e.g. oral administration comprising levodopa (or disclosed ester thereof), an arginine salt of levodopa, carbidopa (or disclosed ester thereof), or an arginine salt of carbidopa, and or a COMT inhibitor and optionally instructions for use.

Also provided herein, in one embodiment, is a method for treatment of a disease or disorder characterized by reduced levels of dopamine in a patients brain, (e.g. Parkinson's disease), comprising substantially continuously administering to a patient in need thereof a therapeutically effective amount of a formulation comprising a disclosed levodopa ester (or salt thereof). Such methods may also include additional administering of an effective amount of carbidopa (or disclosed ester and/or salt thereof) or pharmaceutically acceptable salt thereof, or composition comprising levodopa, e.g., a disclosed salt and/or ester thereof (for example, administering a composition e.g. a tablet, having levodopa as its sole active agent), or a composition that includes carbidopa and one or more other active agents such as levodopa, benserazide, entacapone, tolcapone, selegiline and/or rasagiline. Contemplated methods of treatment included those directed to diseases or disorders including restless leg syndrome, Parkinson's disease, secondary parkinsonism, Huntington's disease, Parkinson's like syndrome, PSP, MSA, ALS, Shy-Drager syndrome, Dystonia and conditions resulting from brain injury including carbon monoxide or manganese intoxication. In an embodiment, continuous administering may include transdermal, intradermal, subcutaneous, intravenous, intramuscular, intrathecal, intratracheal, or intraduodenal administration, e.g. may include the use of an infusion pump.

In one embodiment, a method of treating or ameliorating a neurological or movement disorder in a patient in need thereof is provided comprising: substantially administering a therapeutically effective amount of a composition comprising a levodopa ester or salt thereof, and administering a therapeutically effective amount of a composition comprising carbidopa or a disclosed salt and/or ester thereof. For example, a composition comprising a levodopa ester may be administered substantially continuously and/or a composition comprising carbidopa may be administered at discrete intervals (for example by oral administration one, two, three or more times a day), during the substantially continuous administration of composition comprising levodopa ester, or a composition comprising carbidopa, carbidopa ester or a disclosed salt may be administered substantially continuously.

### BRIEF DESCRIPTION OF THE FIGURES

FIGS. 1A, 1B, and 1C depict mass spectra of carbidopa arginine salt.

FIGS. 2A, 2B, and 2C depict mass spectra of levodopa arginine salt.

FIGS. 3A, 3B, and 3C show the mean levels of carbidopa determined in plasma of female landrace×large white swine (30-35 kg) following oral administration of (FIG. 3A) Stalevo (100/25/200 mg, LD/CD/E), (FIG. 3B) Dopicar+Lodosyn (125/25 mg LD/CD), (FIG. 3C) Sinemet CR (100/25 mg, LD/CD) Q8 and 12 h, with (squares) or without (diamonds) continuous subcutaneous administration of 3% carbidopa solution.

FIGS. 4A and 4B show brain levels of dopamine & L-Dopa (FIG. 4A, top and bottom, respectively), and plasma levels of L-dopa and carbidopa (FIG. 4B, top and bottom, respectively) determined in CD-1 mice following oral administration of levodopa/carbidopa with or without continuous subcutaneous administration of carbidopa.

FIGS. 5A and 5B depict mean levels of L-Dopa & carbidopa determined in plasma of female landrace×large white swine (30-35 kg) following continuous subcutaneous administration of 0, 2 and 4% carbidopa with oral administration of Sinemet® (100/25 mg) q8h.

FIGS. 6A and 6B depict mean levels of L-dopa determined in plasma of female landrace×large white swine (30-35 kg) following continuous subcutaneous administration of 2 and 4% carbidopa with oral administration of Dopicar® (125/12.5 mg LD/CD)+Lodosyn® (12.5 mg CD) q12h.

FIG. 7 shows the mean (±SD) LD (levodopa) concentrations (ng/ml) as determined in plasma of female landrace×large white swine (30-35 kg) following oral administration of Stalevo (LD/CD/E 100/25/200), q8h, with or without continuous subcutaneous benserazide or carbidopa (60 mg/day).

FIG. 8A depicts plasma levels of L-dopa and FIG. 8B depicts plasma levels of 3-O-methyldopa (3-OMD), as determined in plasma of female landrace×large white swine (30-35 kg) following continuous subcutaneous administration of 2% carbidopa, with or without 2.5% entacapone, and oral administration of L-dopa/Carbidopa (LD/CD).

FIGS. 9A and 9B show the results of transdermal delivery of carbidopa propyl ester.

FIG. 10A depicts plasma levels of levodopa and FIG. 10B depicts carbidopa, as determined in plasma of female landrace×large white swine (30-35 kg) following oral administration of LD and CD as arginine salts (designated Ins and CDs, respectively, 100/25 mg LD/CD) as compared to Sinemet (100/25 mg LD/CD).

FIG. 11 depicts the inhibition of L-dopa decarboxylation by carbidopa and carbidopa propyl ester.

FIG. 12A depicts the inhibition of L-dopa decarboxylation by carbidopa propyl ester and FIG. 12B depicts the metabolism of levodopa to dopamine.

FIG. 13 shows LD in plasma after spiking of LD and LD esters into whole blood.

FIGS. 14A, 14B, 14C, and 14D depict results in a pig model after intradermal administration of LD esters for 4 hours.

FIG. 15A shows LD plasma levels in a pig model after IV infusion of LD propyl ester and FIG. 15B shows LD plasma levels in a pig model after IV infusion of LD isopropyl ester.

FIG. 16 shows LD concentration following administration via patch in blood and plasma (left); and 3-OMD and LD concentration in blood and plasma (right).

FIG. 17 shows plasma LD following continuous subcutaneous administration of LD benzyl ester HCl.

FIG. 18 shows LD in plasma after subcutaneous continuous administration of LDE/CDE in pigs.

FIGS. 19A and 19B show the effect of pH on stability and hydrolysis, respectively, of LD isopropyl ester.

FIGS. 20A and 20B show the NMR spectra of LD propyl ester and CD propyl ester, respectively.

### DETAILED DESCRIPTION OF THE INVENTION

Disclosed herein is a liquid aqueous composition having a physiologically acceptable pH that includes a levodopa ester (e.g., a levodopa ester such as, but not limited to methyl ester, ethyl ester, propyl ester, isopropyl ester, benzyl ester, or salt thereof, e.g., but not limited to HCl, tartrate, succinate, fumarate, adipate, aspartate, glutamate salt of levodopa esters) that is stable at room temperature, which can facilitate continuous delivery of an effective amount levodopa to a patient in a minimally invasive fashion (e.g. a disclosed liquid formulation comprises a significantly high concentration of levodopa so that administration of large amounts of liquid are not required). Further, it has been discovered that the pharmacokinetic profile of, for example, levodopa supports such new therapies that include substantially continuous administration of L-dopa together with administration (continuous or at discrete intervals) of e.g. carbidopa or a salt or ester or an ester salt thereof with or without COMT inhibitors.

Disclosed herein is a liquid composition having a physiologically acceptable pH that includes an arginine salt of carbidopa (e.g., arginine and carbidopa) that is stable at room temperature, which can facilitate continuous delivery of an effective amount carbidopa to a patient in a minimally invasive fashion (e.g. a disclosed liquid formulation comprises a significantly high concentration of carbidopa so that administration of large amounts of liquid are not required) Such formulations may facilitate continuous decarboxylase inhibition which prolongs the half-life of levodopa. example, results from in vivo studies, as described below, in which L-dopa ester was administered continuously in parallel with oral administration of carbidopa every 6-8 hours demonstrate a pulsatile pattern of L-dopa plasma levels that coincided with carbidopa oral dosing regimen. In contrast, continuous administration of dopa decarboxylase inhibitor (e.g. of carbidopa or a carbidopa ester of a salt thereof, or benserazide) with or without COMT inhibitors with discrete or continuous administration of levodopa is more effective in the treatment of e.g., Parkinson's disease. Further, it has been discovered that the pharmacokinetic profile of, for example, carbidopa (with or without entacapone) supports such new therapies that include substantially continuous administration of dopa decarboxylase inhibitors (e.g. benserazide or carbidopa or a salt or ester thereof) with or without COMT inhibitors together with administration (continuous or at discrete intervals) of e.g. levodopa or a salt and/or ester thereof.

Provided herein are formulations of levodopa that unexpectedly allow for stable dissolution of higher concentrations of levodopa at e.g. physiologically acceptable pH, for e.g., substantially continuous subcutaneous or transdermal administration. Such formulations may also be suitable for intravenous, intradermal, subcutaneous, transdermal, intrathecal, intratracheal, intranasal, intramuscular, intragastric, oral or intraduodenal administration.

Also provided herein are formulations of carbidopa that unexpectedly allow for stable dissolution of higher concentrations (e.g., greater than about 0.5% or greater than about 1% by weight) of carbidopa and/or higher concentrations (e.g., greater than 2% by weight) levodopa at e.g. physiologically acceptable pH, for e.g., substantially continuous subcutaneous or transdermal administration, Such formulations may also be suitable for intravenous, intramuscular, intrathecal, intranasal, intratracheal, intradermal, oral or intraduodenal administration. For example, provided herein are formulations and methods capable of obtaining substantially constant inhibition of dopa decarboxylase activity upon administration, thereby increasing the half-life of administered levodopa and substantially reducing the pulsatility of levodopa plasma levels to avoid low trough levels of plasma levodopa.

A treatment strategy of continuous carbidopa administration in accordance with the present invention may simulate L-dopa substantially continuously. For example, therapies and/or methods of the present invention may extend a levodopa oral dosing regimen to about 2 to about 3 times/day, and/or reduce daily dose of levodopa, and/or reduce or even eliminate the risk of motor complications associated with standard oral levodopa formulations in Parkinson's patients.

### Compositions

Provided herein, in an embodiment, is a pharmaceutically acceptable formulation that includes a carbidopa salt such as carbidopa arginine, that allows for substantially continuous administration of carbidopa. For example, while carbidopa free base is practically insoluble in alcohol, chloroform or ether and only slightly soluble in water, provided herein, for example, is a stable liquid formulation that includes carbidopa and may be suitable for substantially continuous administration to a patient. Further, such formulations may have a physiologically acceptable pH.

In one aspect, the present invention relates to a carbidopa salt with a basic amino acid selected from arginine, lysine, or histidine. In one preferred embodiment, the salt is the carbidopa arginine salt.

The disclosure also provides, in an embodiment, a liquid formulation comprising a disclosed carbidopa salt. For example, a disclosed carbidopa salt (e.g. carbidopa arginine, carbidopa histidine, carbidopa lysine) may be dissolved in an aqueous solution, (e.g., having a pH of about 6 to 9.5, preferably from about 7 to about 9, more preferably from about 8 to 9 at 25 C or at 30° C. Alternatively, carbidopa (free base) and a basic amino acid salt (e.g. arginine, histidine and/or lysine) are dissolved together in a liquid (e.g. an aqueous liquid) to form a disclosed liquid formulation. Disclosed liquid formulations may include about 1.0% by weight or more carbidopa or carbidopa salt, for example, may include about 1% to about 20% by weight or more carbidopa, e.g., about 2% to about 10% by weight carbidopa. For example, a liquid formulation may include carbidopa and a basic amino acid (such as arginine) in molar ratio of about 1:0.5 to about 1:2.5, or about 1:1 to about 1:1.2, or about 1:1 to about 1:1.5, e.g., about 1:1.2 or 1:1.3.

Also provided herein, in an embodiment, is a pharmaceutically acceptable formulation that includes a carbidopa salt, a carbidopa ester or a salt of a carbidopa ester and levodopa, which allows for substantially continuous administration of levodopa or carbidopa. For example, while levodopa free base is practically insoluble in alcohol, chloroform or ether and only slightly soluble in water, provided herein, for example, is a stable liquid formulation that includes a disclosed levodopa ester and may be suitable for substantially continuous administration to a patient. Further, such formulations may have a physiologically acceptable pH.

Provided herein are carbidopa ester and/or levodopa esters or pharmaceutically acceptable salt thereof, wherein the carbidopa or levodopa ester includes a moiety selected from the group consisting of: —C₁₋₈alkyl (optionally substituted by one or more substituents independently selected from: hydroxyl, phenyl, halogen, or C₁₋₆alkoxy), and (CH₂)₂—(O—(CH₂)₂—)_{q}—, wherein r is an integer from 1 to about 10, e.g., 1, 2 or 3 and q is an integer from about 1 to about 10, e.g., 1, 2, 3, 4, 5, 6, 7, or 8.

The disclosure also provides, in an embodiment, a liquid formulation comprising a. disclosed levodopa ester (or pharmaceutically acceptable salt thereof). For example, a disclosed levodopa ester (e.g. but not limited to levodopa methyl ester, levodopa ethyl ester, levodopa propyl ester, levodopa isopropyl ester, levodopa butyl ester, levodopa hexyl ester, levodopa octyl ester, levodopa isobutyl ester, levodopa propylene glycol ester or pharmaceutically acceptable salts thereof, e.g., but not limited to HCl tartrate, succinate, adipate, fumarate, aspartate, glutamate salt of levodopa ester) may be dissolved in an aqueous solution, (e.g., having a pH of about I to 9.5, or about 4 to about 6, at 25° C. or at 30° C. Disclosed liquid formulations may include about 5%, 10%, 30%, 40%, 50%, 60% (w/v) or more levodopa ester or salt thereof (and/or carbidopa ester or salt thereof), for example, may include about 5% to about 60% (w/v) by weight or more levodopa, e.g., about 10% to about 30% (w/v), about 30% to about 60% ((w/v) by weight levodopa. For example, a liquid formulation may include about 10%-30% (v/v) water and about 25%-35% (w/v) levodopa ester, or about 20%-25% (v/v) water and about 26%-35% (w/v) levodopa ester, or about 60%-75% (v/v) water and about 25%-35% (w/v) disclosed levodopa ester, or about 75%-95% (v/v) water and about 5%-20% (w/v) levodopa ester.

Disclosed liquid formulations (e.g. a liquid composition comprising carbidopa and arginine, an arginine salt of carbidopa, a carbidopa ester or a salt of carbidopa ester with or without a levodopa ester or a salt thereof) may be stable for 24 hours, for 48 hours, for 7 days, or more at 25° C. For example, an exemplary liquid formulation may include a 1:1.2 molar ratio of carbidopa:arginine, with about 1% to about 15%, or about 2% to about 10%, or 0.6% to about 6% by weight carbidopa. Disclosed liquid formulation may be more stable (e.g., substantially free of precipitation and/or the carbidopa or levodopa has undergone minimal degradation) at 7 days, or 30 days or more as compared to another liquid composition that includes e.g., a lysine or histidine salt of carbidopa.

In some embodiments, disclosed liquid formulations or compositions are liquid solutions, i.e. are substantially homogenous liquid mixtures. Such liquid mixtures may comprise water and/or other excipients. In another embodiment, disclosed liquid compositions may be substantially non-aqueous.

For example, as disclosed in Example 6, below, a stable liquid solution can be unexpectedly formed from carbidopa and arginine. Such a solution is stable at room temperature, e.g., is a substantially clear solution, even at high carbidopa concentrations of 2, 3, 4, 6, and/or 8 weight percent carbidopa. Such solutions are stable (e.g., no precipitation) at least for 48 hours. Further, because such disclosed solutions, even at high concentrations of carbidopa, or levodopa (e.g. a salt and/or ester thereof), have a physiologically acceptable pH, such solutions can be adjusted to an appropriate pH, but still have a significant amount of carbidopa in a smaller volume so that it facilitates patient administration, without e.g. administering large volumes of solution.

Further, solutions having carbidopa and arginine (e.g., the arginine salt of carbidopa) are unexpectedly more stable even as compared to solutions of carbidopa with histidine or lysine, as shown below in e.g. Example 6.

Contemplated liquid formulations having e.g. carbidopa arginine may, in some embodiments, further comprise levodopa, levodopa ester or levodopa and arginine, and/or optionally a catechol-O-methyl transferase (COMT) inhibitor, such as entacapone or tolcapone; and/or a monoamine oxidase (MAO)-A or MAO-B inhibitor, such as moclobemide, rasagiline, selegiline or safinamide. Contemplated liquid formulations having e.g. levodopa ester may, in some embodiments, further comprise carbidopa, carbidopa and arginine, carbidopa ester or salt of a carbidopa ester (e.g. but not limited to, carbidopa methyl ester, carbidopa ethyl ester, carbidopa propyl ester, carbidopa isopropyl ester, carbidopa butyl ester, carbidopa hexyl ester, carbidopa octyl ester, carbidopa isobutyl ester, or pharmaceutically acceptable salts thereof, e.g. but not limited to HCl, tartrate, succinate, fumarate adipate, aspartate, glutamate salt of carbidopa ester) or carbidopa and arginine, and/or optionally a catechol-O-methyl transferase (COMT) inhibitor, such as entacapone or tolcapone; and/or a monoamine oxidase (MAO)-A or MAO-B inhibitor, such as moclobemide, rasagiline, selegiline or safinamide.

Also disclosed herein is a levodopa salt with a basic amino acid selected from the group consisting of arginine, lysine, and histidine, for example, an arginine salt of levodopa. For example, provided herein is a liquid formulation comprising an arginine salt of levodopa, or a liquid formulation comprising arginine and levodopa. In an embodiment, provided herein is a liquid formulation that includes levodopa and arginine in a molar ratio of about 1:1.5 to about 1:3.5, or about 1:2 to about 1:2.5. Such levodopa and arginine formulations or solutions may have a pH of about 8 to about 10, for example, about 8.5 to about 9.5, or about 9.1 to about 9.8 at 25° C. A disclosed formulation having levodopa and arginine may include about 2%, 3%, 4%, 5%, 6%, 7%, 8%, 9%, 10%, 11%, 12% or more by weight levodopa, e.g., may include about 4% or more by weight levodopa.

In some embodiments, a disclosed liquid formulation will be stable for a period of I day, 2 days, 3 days, 1 week, or 1 month or more at room temperature. In preferred embodiments of the invention, a disclosed liquid formulation further comprise a pharmaceutically acceptable excipient such as e.g., N-methylpyrrolidone (NMP), or polyvinylpyrrolidone (PVP), or both, and/or may further comprise one or more antioxidants such as, but not limited to, N-acetyl cysteine, L-cysteine, sodium bi sulfite, glutathione, ascorbic acid, sodium ascorbate or Vitamin E. For example, in one embodiment, provided herein is a stable liquid formulation that comprises about 0.5 to about 20% of carbidopa (e.g. about 1% or about 2% to about 6%), about 1 to about 20% arginine, about 0 to about 30% NMP, about 0 to about 5% PVP, and/or about 0 to about 5% of one or more water soluble antioxidants, by weight. For example, in another embodiment, provided herein is a stable liquid formulation that comprises about 0.5 to about 20% of levodopa or ester thereof (e.g. about 1% or about 2% to about 40%), about 0 to about 35% arginine, about 0 to about 30% NMP, about 0 to about 5% PVP, and/or about 0 to about 5% of one or more water soluble antioxidants, by weight.

The invention further provides a stable lyophilized powder comprising a disclosed carbidopa salt. In one embodiment, such stable lyophilized powder may comprise about 20-99% of the carbidopa salt, about 0-60% NMP, about 0-15% PVP, and about 0-5% of one or more water soluble anti oxidants. The lyophilized powder can be reconstituted into a liquid formulation by addition of water alone or water with NMP, and may include or not include antioxidants.

Liquid formulations of the invention may be designed for continuous administration of a carbidopa (or ester or salt thereof) with or without levodopa (or ester or salt thereof) to a patient in need thereof. For example, a patient may be substantially continuously administered (e.g. subcutaneously, transdermally, intraduodenally, intradermally, intragastrically, intratechecally or intravenously) a formulation that includes a disclosed carbidopa salt such as the arginine salt of carbidopa, while levodopa, a levodopa salt, or a composition comprising levodopa is orally administered at discrete intervals, e.g., 2, 3, 4, or 5 times a day.

As used herein in the specification, the term “a composition comprising levodopa” contemplates formulations that comprise levodopa, levodopa salt, levodopa ester, or salt of a levodopa ester optionally together with a decarboxylase inhibitor, a catechol-O-methyl transferase (COMT) inhibitor, and/or a MAO-A or MAO-B inhibitor. For example, a composition comprising levodopa includes a dosage formulation that comprises levodopa (or an ester and/or salt thereof) and optionally another drug, where the dosage formulation may be an immediate release, controlled release, dual release or multiple release formulation suitable for oral administration.

The term “decarboxylase inhibitor” refers to a dopa decarboxylase inhibitor, e.g., a drug that inhibits the peripheral metabolism of levodopa to dopamine by aromatic L-amino acid decarboxylase such as carbidopa (or ester or salt thereof) and benserazide.

The term “pharmaceutically acceptable carrier” or “pharmaceutically acceptable excipient” as used herein refers to any and all solvents, dispersion media, preservatives, antioxidants, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “physiologically acceptable pH” is understood to mean a pH of e.g., a composition that facilitates administration of the composition to a patient without significant adverse effects, e.g. a pH of about 3.5+0.5 to about 9.5+0.5.

COMT inhibitors refer to inhibitors that inhibit the degradation of levodopa to 3-methyldopa by catechol-O-methyl transferase and prolong the action of levodopa, such as entacapone or tolcapone. For example, compositions comprising levodopa contemplated herein may also include a decarboxylase inhibitor (carbidopa or benserazide) and entacapone, e.g. “triple therapy”.

MAO-A or MAO-B inhibitors prevent the breakdown of dopamine by monoamine oxidases, e.g., moclobemide, rasagiline, selegiline or safinamide, more preferably, rasagiline.

Also contemplated herein is a kit comprising: a) a first formulation comprising a carbidopa salt and/or carbidopa and arginine or a carbidopa ester and or salt of carbidopa ester, with or without levodopa salt and/or levodopa and arginine or a levodopa ester and or salt of levodopa ester wherein said first formulation is suitable for continuous administration; b) a second formulation comprising levodopa, levodopa ester, or an arginine salt of levodopa, and/or a COMT inhibitor wherein said second formulation is suitable for oral administration; and c) instructions for administration of formulation a) in conjunction with formulation b). The formulation a) comprising the carbidopa salt may be suitable for continuous administration by any suitable route such as transdermally, intravenously, subcutaneously, intradermally, intramuscularly, intragastric or intraduodenally.

The first formulation of a contemplated kit comprising the carbidopa salt or carbidopa ester may be liquid or a lyophilized powder that can be reconstituted into a liquid formulation, or, for example, may form part of a transdermal patch, and may be designed for continuous administration by any suitable route such as, but not limited to, transdermally, intravenously, subcutaneously, intradermally, intramuscularly, intragastric or intraduodenally. In an embodiment, the first formulation comprises a disclosed carbidopa salt or ester and is suitable for administration subcutaneously. The second formulation of a contemplated kit may include the levodopa, a levodopa ester, a levodopa salt, a levodopa ester salt or a composition comprising levodopa, and may be presented as any suitable oral dosage such as, but not limited to, pills, tablets, dispersible tablets, capsules, liquid, and the like. In an embodiment, the second formulation may be in the form of an immediate release, controlled release or dual release oral formulation that comprises both levodopa and benserazide, or both levodopa and carbidopa(or salt or ester thereof). Such oral formulation in the form of pills, tablets, or the like, may comprise a ratio of carbidopa or benserazide to levodopa of about 1:10 to 1:4, or from about 1:4 to 1:1. Other contemplated second formulations include formulations, e.g., tablets that include levodopa, carbidopa, and entacapone, or e.g. a tablet that includes levodopa arginine salt or levodopa ester and/or carbidopa arginine salt or carbidopa ester. In another embodiment, the second formulation may be or includes a COMT inhibitor.

In another embodiment, the first formulation of a contemplated kit comprises a carbidopa (or salt or ester) with a levodopa salt or ester (e.g., a carbidopa ester with a levodopa ester or salts thereof), wherein the formulation may be liquid or a lyophilized powder that can be reconstituted into a liquid formulation, or, for example, may form part of a transdermal patch, and may be designed for continuous administration by any suitable route such as, but not limited to, transdermally, intravenously, intrathecally, intragastric, subcutaneously, intradermally, intramuscularly or intraduodenally. In an embodiment, the first formulation comprises a disclosed carbidopa salt with levodopa salt or carbidopa ester with levodopa ester or salts thereof and is suitable for administration subcutaneously. The second formulation of a contemplated kit may include a levodopa, a levodopa ester, a levodopa salt, or a composition comprising levodopa, or a COMT inhibitor and may be presented as any suitable oral dosage such as, but not limited to, pills, tablets, dispersible tablets, capsules, liquid, and the like. In an embodiment, the second formulation may be in the form of an immediate release, controlled release or dual release oral formulation that comprises both levodopa and benserazide, or both levodopa and carbidopa. Such oral formulation in the form of pills, tablets, or the like, may comprise a ratio of carbidopa or benserazide to levodopa of about 1:10 to 1:4, from about 1:4 to 1:1. Other contemplated second formulations include formulations, e.g., tablets that include levodopa, carbidopa, and entacapone, or e.g. a tablet that includes levodopa arginine salt or ester and/or carbidopa arginine salt or ester.

In another embodiment, the kit comprises carbidopa and arginine, a first liquid formulation comprising levodopa ester suitable for, but not limited to, transdermal, intravenous, subcutaneous, intradermal, intramuscular, intraduodenal, intragastric continuous administration, and optionally a second formulation in the form of an immediate release, controlled release or dual release oral formulation comprising levodopa and/or carbidopa, and/or entacapone. The oral formulation in the form of pills, tablets, or the like, may comprise a ratio of carbidopa to levodopa from about 1:10 to about 1:4, preferably from about 1:4 to about 1:1.

In another aspect, the present invention relates to a formulation comprising a carbidopa ester such as, but not limited to, the ethyl, propyl, isopropyl or hexyl ester of carbidopa, and salts thereof. Examples of levodopa esters contemplated herein include the alkyl esters, e.g., the methyl, ethyl, propyl, or isopropyl ester, or the benzyl ester.

In another aspect, the present invention relates to a formulation comprising a levodopa ester such as, but not limited to, the ethyl, propyl, isopropyl or hexyl ester of levodopa, and salts thereof. Examples of levodopa esters contemplated herein include the alkyl esters, e.g., the methyl, ethyl, propyl, or isopropyl ester, or the benzyl ester.

### Methods

In a further aspect, the present invention provides a method for treatment of a disease or disorder characterized by reduced and/or fluctuating levels of dopamine in a. patient's brain, comprising administering substantially continuously to a patient in need a therapeutically effective amount of L-dopa and/or a decarboxylase inhibitor, or a salt and/or an ester thereof. For example, contemplated method of administering substantially continuously an L-dopa or may include administration of a therapeutically effective amount of carbidopa, carbidopa ester or salts thereof.

Transdermal delivery is one way of providing L-dopa to the blood circulation continuously. However, L-dopa itself is a problematic candidate for transdermal delivery due to its low solubility, instability in solution and poor skin permeability. L-dopa prodrugs or salts (e.g. a levodopa ester as disclosed herein), with higher solubility than L-dopa, may constitute better candidates for transdermal delivery.

As shown in the Examples, separate continuous administration of carbidopa , together with administration of levodopa, even with discrete (e.g. oral) administration of levodopa, to a patient results in significantly higher levels of levodopa in the plasma of a patient upon administration as compared to a current standard of discrete carbidopa and. levodopa simultaneous dosing. For example, disclosed methods may result in a half-life of levodopa in the plasma of a patient that is at least 1.25, or at least two times, longer after continuous administration of carbidopa as compared to the half life of levodopa in a patient's serum after administering levodopa without continuous administration of carbidopa (e.g., with discrete, oral administration).

Contemplated administration of e.g., carbidopa. and/or levodopa, following the disclosed methods, typically can be carried out over a defined time period (usually weeks, months or years depending upon the combination selected). Contemplated therapies are intended to embrace administration of multiple therapeutic agents in a manner wherein a dopa decarboxylase inhibitor is administered substantially continuously, while levodopa, levodopa ester or salt thereof is administered at discrete intervals and/or substantially continuously, as well as administration of contemplated therapeutic agents, or at least two of the therapeutic agents, in a substantially simultaneous manner. Contemplated therapies are also intended to embrace administration of multiple therapeutic agents in a manner wherein a levodopa ester is administered substantially continuously while carbidopa is optionally administered at discrete intervals, (or carbidopa is substantially continuously administered) Administration can be effected by any appropriate route including, but not limited to, oral routes, intravenous routes, intramuscular routes, intradermal routes, subcutaneously, transdermally, intrathecally, intragastric, intraduodenally and direct absorption through mucous membrane tissues.

In some embodiments, levodopa, levodopa salt, levodopa ester or salt thereof can be administered by the same route or by different routes as compared to administration of e.g. a contemplated carbidopa formulation. For example, carbidopa salt, carbidopa ester or salt thereof may be administered subcutaneously, e.g., substantially continuously, while levodopa, levodopa salt, levodopa ester or a salt thereof may be administered orally, e.g. at discrete intervals. Alternatively, levodopa salt, levodopa ester or salt thereof may be administered subcutaneously, e.g., substantially continuously, while carbidopa, carbidopa salt or carbidopa ester may be administered orally, e.g. at discrete intervals. In an embodiment, a disclosed liquid carbidopa. composition (e.g. having carbidopa and arginine) is administered substantially continuously, while an oral composition that includes levodopa (and may also include one or more other active agents such as a dopa decarboxylase inhibitor) is administered at discrete intervals. Alternatively, for example, levodopa and/or carbidopa salts, esters or salts thereof may be administered subcutaneously or transdermally.

The disease or disorder characterized by reduced levels of dopamine in the brain contemplated herein are neurological or movement disorders including, but not limited to, restless leg syndrome, Parkinson's disease, secondary parkinsonism, Huntington's disease, Shy-Drager syndrome, dystonia and conditions resulting from brain injury including carbon monoxide or manganese intoxication. In one preferred embodiment, the disease to be treated is Parkinson's disease.

In preferred embodiments, the contemplated decarboxylase inhibitor is the arginine salt of carbidopa. A disclosed carbidopa/arginine formulation may be administered substantially continuously using e.g. a liquid formulation, for example, via a pump for subcutaneous infusion (insulin pump) at an average rate of about 10-250 μl/hour, preferably about 5-85 μl/hour, in conjunction with oral administration of levodopa, levodopa ester, an arginine salt of levodopa, or composition comprising levodopa.

For example, a method for treating a neurological or movement disorder e.g., Parkinson's disease, is provided herein comprising substantially continuously administering to a patient in need thereof a pharmaceutically effective amount of a composition comprising a carbidopa and an amino acid such as arginine, lysine or histidine, and administering a pharmaceutically effective amount of composition comprising levodopa. For example, the composition comprising a carbidopa and arginine may be liquid at room temperature. The disclosed composition may be administered substantially continuously over 12 hours, 16 hours, 1 day, 1 week, or more. The composition comprising levodopa may form all or part of an immediate release, controlled release, or dual release oral formulation comprising levodopa and optionally benserazide or carbidopa, and may be administered 1, 2, 3, or 4 times a day, for example, by oral administration (e.g. by tablet).

Also provided herein is a method for treatment of a disease or disorder characterized by reduced levels of dopamine in a patient's brain, (e.g., Parkinson's disease) comprising co-administering substantially continuously to a patient in need a therapeutically effective amount of a disclosed levodopa salt and/or ester (or a disclosed formulation thereof).

The invention now being generally described, it will be more readily understood by reference to the following examples which are included merely for purposes of illustration of certain aspects and embodiments of the present invention, and are not intended to limit the invention in any way.

### EXAMPLES

### Example 1

### Preparation and Characterization of Carbidopa-Arginine Salt

Carbidopa-Arginine salt was prepared as follows:

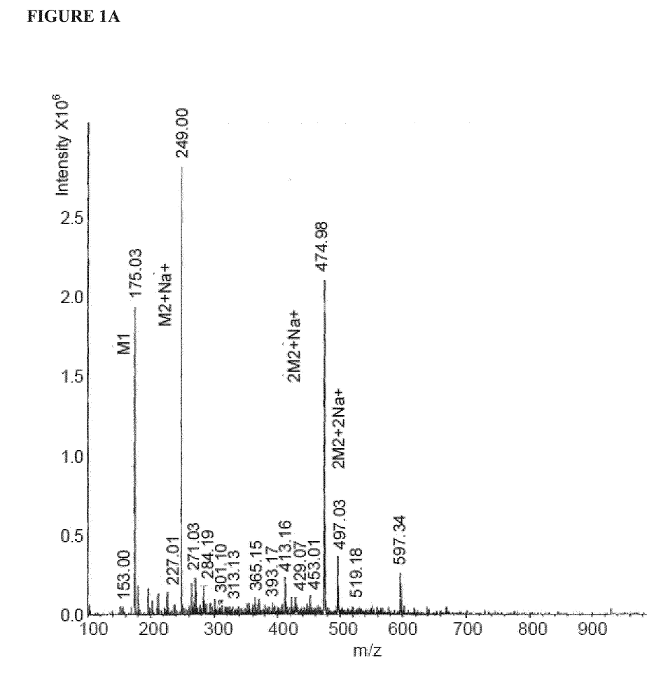

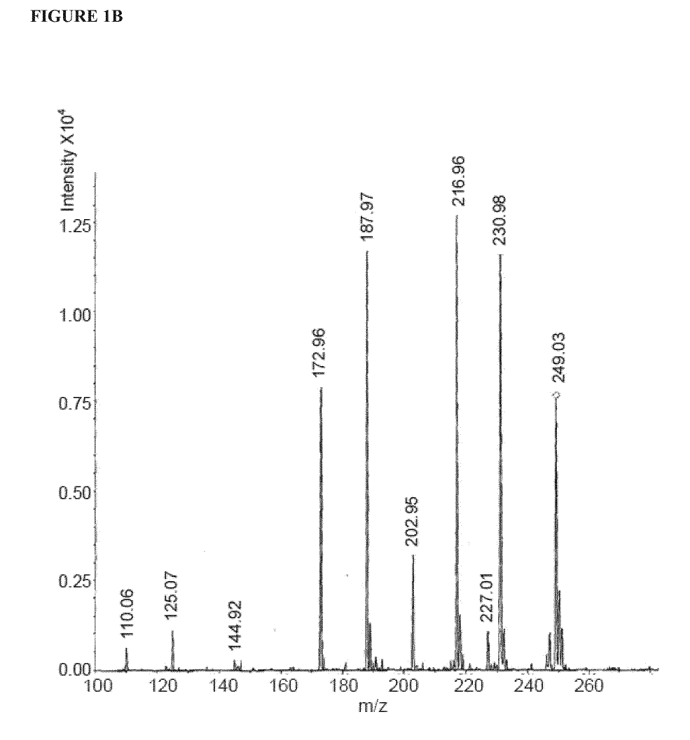

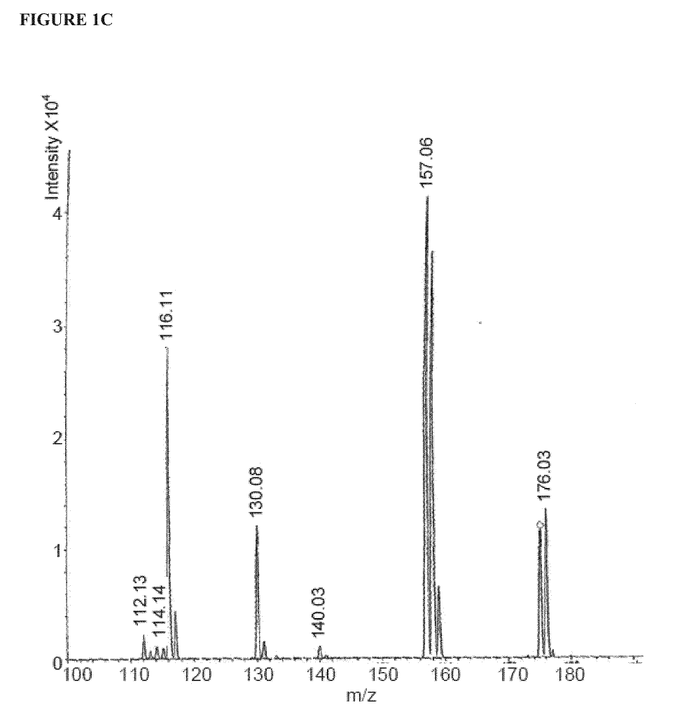

Carbidopa [TEVA] was weighed in a suitable container with L-arginine [MERCK] (at molar ratio of 1:1) and a 0.2% sodium bisulfite [SIGMA] solution in water was added to obtain a final concentration of 4.0% carbidopa. The mixture was heated to 65±10° C. with constant stirring. When the solids were completely dissolved, solution was filtered using 0.45 nylon membrane. The filtered solution was immediately frozen in dry ice and subsequently subjected to lyophilyzation. Off-white crystals were obtained and subsequently subjected to MS analysis. The MS analytical results clearly showed carbidopa and L-arginine ions and fragments (FIG. 1A). Peak 249 represents carbidopa+Na (226+23) with fragments: 227, 188 & 144 (FIG. 1B); Peak 176 represents arginine+2H (174+2) with fragments: 157,130 & 116 (FIG. 1C).

### Example 2

### Preparation of Carbidopa Solution/Formulation for Subcutaneous Administration

A 4% Carbidopa solution/formulation was prepared as follows:

Carbidopa [ASSIA Ltd.] was weighed in a suitable container and water was then added to obtain 73% of the total projected batch weight. Mixture was stirred at room temperature for 20 minutes. L-Arginine [Sigma] was added to the mixture to obtain a molar ratio 1:1 with Carbidopa. The mixture was heated to 65±10° C. with constant stirring. When the solids were completely dissolved, N-methyl 2-pyrrolidone [Pharmasolve, ISP] was added to obtain the final concentration of 10% (w/w ). Sodium bisulfite [Sigma] solution was prepared and added to obtain a final concentration of 1% (v/w). Stirring was continued for additional 30 minutes at 65±3° C. Thereafter, PVP [Polyvinylpyrrolidone, Sigma] solution was prepared and added to obtain a final concentration of 1% (v/w). Stirring was continued for 30 minutes at 65±3° C. Heating was stopped and the preparation was allowed to cool down to room temperature. Solution was filtered using a sterile 0.22 μM PVDF membrane.

Carbidopa-Arginine solutions/formulations, 2 and 3%, were prepared by diluting the 4% Carbidopa-arginine solution/formulation with the respective amount of double distilled water (DDW).

### Example 3

### Preparation of Carbidopa Solution/Formulation for Subcutaneous Administration

A 6% Carbidopa solution/formulation was prepared as follows:

Carbidopa [TEVA] and L-arginine [MERCK] (molar ratio 1:1.1) were weighed in a suitable container and water was then added to obtain 84% of the total projected batch weight. N-methyl 2-pyrrolidone [Pharmasolve, ISP] was added to obtain the final concentration of 5% (w/w) Sodium bisulfite [SIGMA] solution was prepared and added to obtain a final concentration of 0.1% (v/w). The mixture was heated to 65±10° C. with constant stirring. When the solids were completely dissolved heating was stopped and the preparation was allowed to cool down to room temperature. Solution was filtered using a sterile 0.22 μM PVDF membrane.

### Example 4

### Preparation of Carbidopa Solution/Formulation for Subcutaneous Administration

A 4% Carbidopa solution/formulation was prepared as follows:

Carbidopa [TEVA] and L-arginine [MERCK] (molar ratio 1:1.1) were weighed in a suitable container and water was added to obtain 89% of the total projected batch weight. N-methyl 2-pyrrolidone [Pharma.solve, ISP] was added to obtain the final concentration of 3.5% (w/w). Sodium bisulfite [SIGMA] solution was prepared and added to obtain a final concentration of 0.05% (v/w). The mixture was heated to 65±10° C. with constant stirring. When the solids were completely dissolved, heating was stopped and the preparation was allowed to cool down to room temperature. The solution was filtered using a sterile 0.22 μM PVDF membrane.

Carbidopa-Arginine solutions/formulations, 2 and 3%, were prepared by diluting the 4% Carbidopa-arginine solution/formulation with the respective amount of double distilled water (DDW) containing 3.5% N-MP, with or without 0.05% sodium bisulfite.

### Example 5

### Preparation of Carbidopa Formulation for Transdermal Delivery

An 8% Carbidopa formulation was prepared as follows:

Carbidopa [TEVA] and L-arginine [MERCK] (molar ratio 1:1) were weighed in a suitable container and propylene glycol [MERCK] was added to obtain 75% of the total projected batch weight. Sodium bisulfite [SIGMA] solution was prepared and added to obtain a final concentration of 0.05%. The mixture was heated to 65±10° C. with constant stirring. When the solids were completely dissolved, heating was stopped and the preparation was allowed to cool down to room temperature. PEG-400 [MERCK]. 10% of the total projected batch weight, was added. The pH was adjusted to 7.5 with 85% lactic acid [FLUKA].

### Example 6

### Preparation and Stability of Carbidopa-Arginine Carbidopa-Lysine and Carbidopa-Histidine Solutions/Formulations

Carbidopa solutions/formulations were prepared as follows:

Carbidopa [TEVA] was weighed in a suitable container with L-arginine [MERCK] or L-Lysine [SIGMA] or L-Histidine [SIGMA] (at molar ratio of 1:1, 1:1.1 or 1:2) and water was added. N-methyl 2-pyrrolidone [Pharmasoive, ISP] was added to obtain the final concentration of 5% (w/w). Sodium bisulfite [SIGMA] solution was prepared and added to obtain a final concentration of 0.05% (v/w). The mixture was heated to 68±3° C. with constant stirring. When the solids were completely dissolved, heating was stopped and the preparation was allowed to cool down to room temperature. Stable formulations (2% CD:Lysine and 2% CD:Arginine 1:1.1 molar ratio) were further subjected to HPLC analysis at t=0 and t=7 days at 25° C.

The results show the significant difference between the three basic amino acids [L-Arginine (P1-10.76), L-Lysine (PI-9.74) and Histidine (P1-7.59)] with respect to their effect on the solubility and stability of carbidopa in aqueous solution: Table 1 indicates the solubility and stability of carbidopa in these aqueous solutions with basic amino acids (arginine, lysine or histidine) as determined visually (Table 1A) or by UV HPLC (Table 1B). With arginine, a stable solution of 6% carbidopa was prepared, whereas a solution with only less than 4% could be formulated with lysine (Table 1A). Furthermore, a solution of 2% carbidopa with lysine was less stable than with arginine after 7days at 25° C. (Table 1B). In addition, a stable solution with histidine at concentrations >1% could not be made (Table 1A).

TABLE 1A
Carbidopa and Arginine Solution

| | | | | | | |
|---|---|---|---|---|---|---|
| CD | 2 | 4 | 4 | 5 | 6 | 8 |
| Concentration | | | | | | |
| (%) | | | | | | |
| Molar Ratio | 1 to 1 | 1 to 1 | 1 to 1.1 | 1 to 1.1 | 1 to 1.1 | 1 to 1.1 |
| CD:Arginine | | | | | | |
| PH of the | 8.2 | 8.2 | 8.4 | 8.5 | 8.7 | 8.9 |
| Solution | | | | | | |
| Solution | Clear, | Clear, | Clear, | Clear; | Clear, | Clear, |
| Appearance | slightly | slightly | slightly | slightly | slightly | slightly |
| | yellow | yellow | yellow | yellow | yellow | yellow |
| Stability after | Stable | Stable | Stable | Stable | Stable | Precip- |
| 48 h (visual) | | | | | | itated |
| | | | | | | within |
| | | | | | | 24 h |

Carbidopa and lysine solutions

| | | | | |
|---|---|---|---|---|
| CD | 2 | 4 | 2 | 4 |
| Concentration | | | | |
| (%) | | | | |
| Molar Ratio | 1 to 1 | 1 to 1 | 1 to 1.1 | 1 to 1.1 |
| CD:Lysine | | | | |
| PH of the | 8.1 | N/A | 8.2 | 8.23 |
| Solution | | | | |
| Solution | Clear, slightly | Didn't | Clear, | Precipitated |
| Appearance | yellow | dissolve | yellow | within few |
| | | | | minutes |
| Stability after | Precipitated | N/A | Stable | N/A |
| 48 h (visual) | after 2 h | | | |

Carbidopa and histidine solutions

| | | | | |
|---|---|---|---|---|
| CD | 1 | 4 | 2 | 4 |
| Concentration | | | | |
| (%) | | | | |
| Molar Ratio | 1 to 1.1 | 1 to 1.1 | 1 to 2 | 1 to 2 |
| CD:Histidine | | | | |
| PH of the | N/A | N/A | 6.7 | N/A |
| Solution | | | | |
| Solution | Didn't | Didn't | Clear, white | Didn't dissolve |
| Appearance | dissolve | dissolve | | |
| Stability after | N/A | N/A | Precipitated | N/A |
| 48 h (visual) | | | after 1 h | |

TABLE 1B

| Amino | Molar | | | CD | Impurities Profile (Area %) | | | | |
|---|---|---|---|---|---|---|---|---|---|
| Acid | Ratio | CD | Time of | Assay | 3- | RT | RT | RT | RT |
| (AA) | CD:AA | (%) | Analysis | (%) | OMD | 5.3 | 12.6 | 13.6 | 14,5 |
| Lysine | 1:1.1 | 2 | t = 0 | 95.1 | 0 | 3.3 | 0 | 0.50 | 1.07 |
| Arginine | 1:1.1 | 2 | t = 0 | 94.1 | 0 | 4.5 | 0 | 0.41 | 1.05 |
| Lysine | 1:1.1 | 2 | t = 7 d | 70.8 | 0 | N/A | 0 | 1.39 | 26.4 |
| | | | at RT | | | | | | |
| Arginine | 1:1.1 | 2 | t = 7 d | 77.6 | 0 | N/A | 0 | 1.34 | 19.4 |
| | | | at RT | | | | | | |

N/A = Not Applicable

### Example 7

### Preparation of Levodopa-Arginine Salt

Levodopa-Arginine salt was prepared as follows:

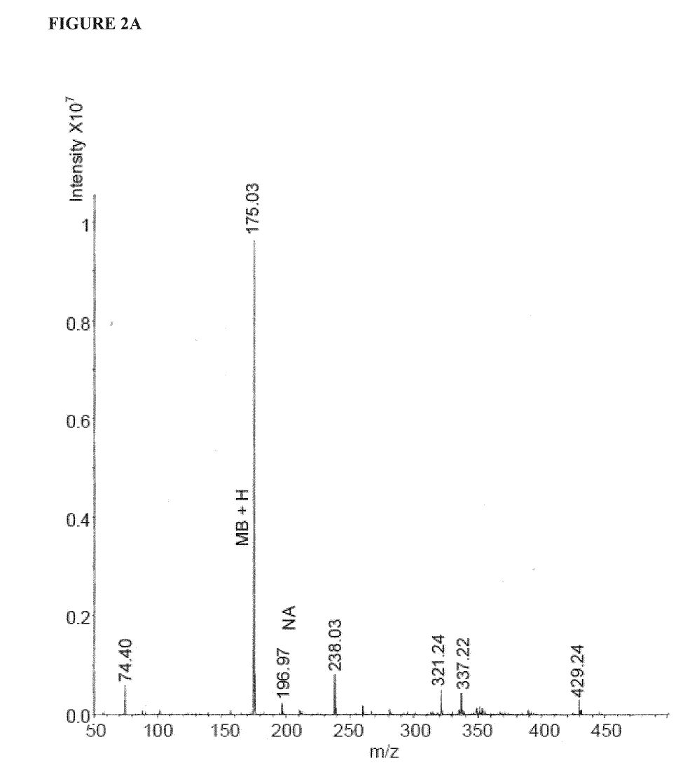

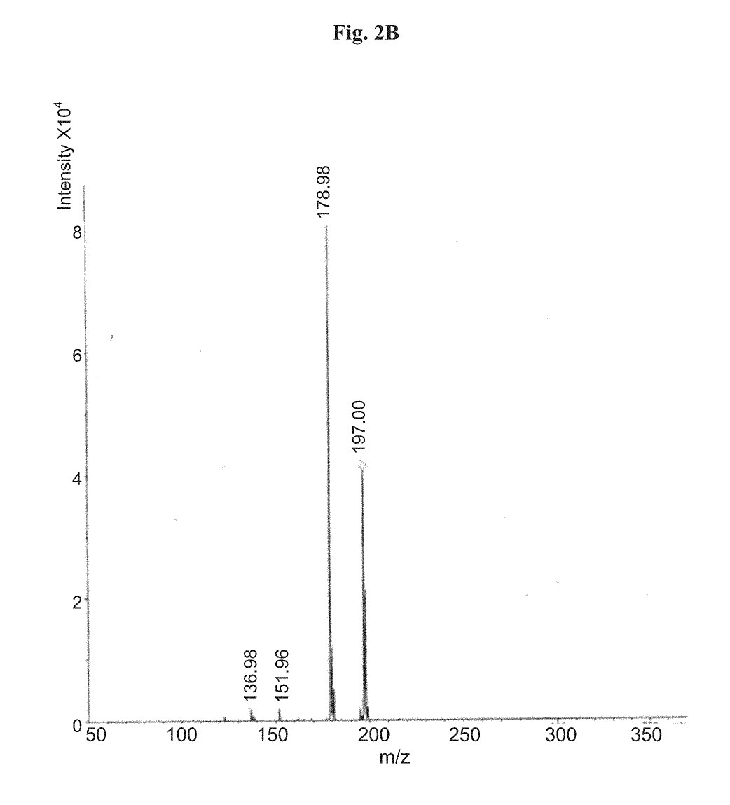

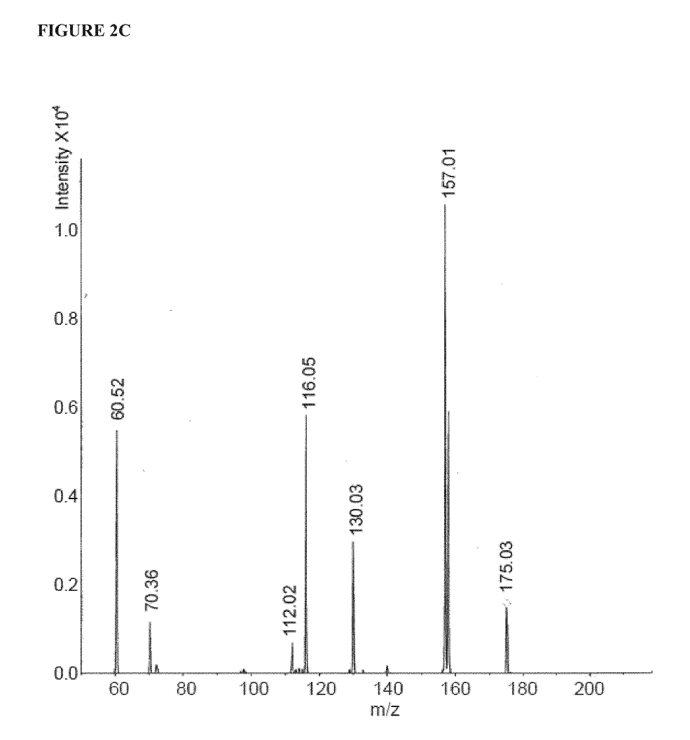

Levodopa [TEVA] was weighed in a suitable container with L-arginine [MERCK] (at molar ratio of 1:1.8) and a 0.2% sodium bislfite [SIGMA] solution in water was added to obtain a final concentration of 4.4% L-Dopa. The mixture was heated to 65±10° C. with constant stirring. When the solids were completely dissolved, solution was filtered using 0.45 μM nylon membrane. The filtered solution was immediately frozen in dry ice and subsequently subjected to lyophilization. The filtered solution was immediately frozen in dry ice and subsequently subjected to lyophilyzation. Off-white crystals were obtained and subsequently subjected to MS analysis. The MS analytical results (shown in FIGS. 2A-2C) clearly showed LD and Arginine ions. LD: 197 with fragments 178.97, 151.96, 136.98 (FIGS. 2A & 2B); Arginine: 175 with fragments 130, 116 (FIGS. 2A & 2C).

### Example 8

### Preparation of Carbidopa and Carbidopa/Entacapone Solutions/Formulations for Subcutaneous Administration, and Their Local Safety Evaluation in Pigs

A 10% carbidopa and 4/6% carbidopa/entacapone solutions/formulations were prepared as follows:

Carbidopa [ASSIA Ltd.] was weighed in a suitable container and water was then added to obtain 73% of the total projected batch weight. Mixture was stirred at room temperature for 20 minutes. L-Arginine [Sigma] was added to the mixture to obtain a molar ratio 1:1 with Carbidopa. The mixture was heated to 65±10° C. with constant stirring. When the solids were completely dissolved, N-methyl 2-pyrrolidone [Pharmasolve, ISP] was added to obtain the final concentration of 10% (w/w). Sodium bisulfite [Sigma] solution was prepared and added to obtain a final concentration of 1% (v/w). Stirring was continued for additional 30 minutes at 65±3° C. Thereafter, PVP [Polyvinylpyrrolidone, Sigma] solution was prepared and added to obtain a final concentration of 1% (v/w), Stirring was continued for 30 minutes at 65±3° C. Heating was stopped and the preparation was allowed to cool down to room temperature. Solution was filtered using a sterile 0.22 μM PVDF membrane. The filtered solution was immediately frozen in dry ice and subsequently subjected to lyophilization. Lyophilized crystals were re-constituted with double distilled water to obtain 4 and 10% carbidopa solutions, Entacapone [extracted from Comtan®, Novartis] was added to the 4% carbidopa solution to obtain a final concentration of 6% (w/v). Both formulations (10% CD and 4/6% CDT) were continuously administered sc to pigs for a period of 21 h to evaluate potential local reactions. Macroscopic and microscopic evaluations indicated that 21 h continuous subcutaneous administration of these carbidopa solutions/formulations was safe. (Table 2).

Table 2 indicates the results of a histological evaluation of skin biopsies obtained from female landrace×large white swine following continuous subcutaneous administration of 10% CD (carbidopa) or 4/6% CD/entacapone for a Period of 21 h, at a Rate of 25 or 82 μl/h

TABLE 2

| | Pig #1 | | | | Pig #2 | | | |
|---|---|---|---|---|---|---|---|---|
| | | | R | | L | | | |
| | L | | CD/Ent | | CD/Ent | | R | |
| | CD 10% | | 4/6% | | 4/6% | | CD 10% | |
| Infusion Rate | Pod Activation (h) | | | | | | | |
| 25 μl/hr | 21 h | | — | | 21 h | | — | |
| 82 μl/hr | — | | 21 h | | — | | 21 h | |
| | Histological Observation (Time post patch removal) | | | | | | | |
| Parameter | 0 | 10 d | 0 | 10 d | 0 | 10 d | 0 | 10 d |
| Lesion | Subcutis | No | No | No | No | No | Subcutis | No |
| Localization | | Le- | Le- | Le- | Le- | Le- | | Le- |
| Distribution | Peri- | sions | sions | sions | sions | sions | Peri- | sions |
| | vascular | | | | | | vascular | |
| Inflammation | 0-1 | | | | | | 0-1 | |
| Grade | | | | | | | | |
| Predominant | N | | | | | | N | |
| Cell Type | | | | | | | | |
| Necrosis | — | | | | | | — | |
| Fibrosis | — | | | | | | — | |

Key:
Localization: Epidermis, dermis, subcutis;
Distribution: Diffuse, multifocal, perivascular;
Inflammation Scoring Grade: 0—no inflammation, 1—very mild, 2—moderate, 3—severe;
Predominant Cell Type: Lymphocytes (L), macrophages (M) neutrophils (N);
Necrosis: Yes/No;
Fibrosis: Yes/No

### Example 9

### The Effect of Continuous Subcutaneous Carbidopa Administration on the Pharmacokinetic Profile of Levodopa and Carbidopa in Pigs

In this experiment, the purpose was to determine the effect of continuous subcutaneous administration of carbidopa, with co-administration of oral L-dopa/carbidopa, on the pharmacokinetics of levodopa in pigs.

Pigs weighing 30-35 kg were administered orally with either Stalevo® (Novartis, 100/25/200 mg, LD/CD/E), Dopicar® [Teva] Lodosyn® (Merck &. Co) (125/25 mg, LD/CD) or Sinemet CR® (MSD,100/25 mg, LD/CD) thrice or twice daily (q8 or 12 h, respectively) with or without carbidopa (60 μmg/pig/d) for a total period of 68 h. Blood samples were collected at pre-determined time points and plasma levels of L-dopa and carbidopa were analyzed by LC-MS.

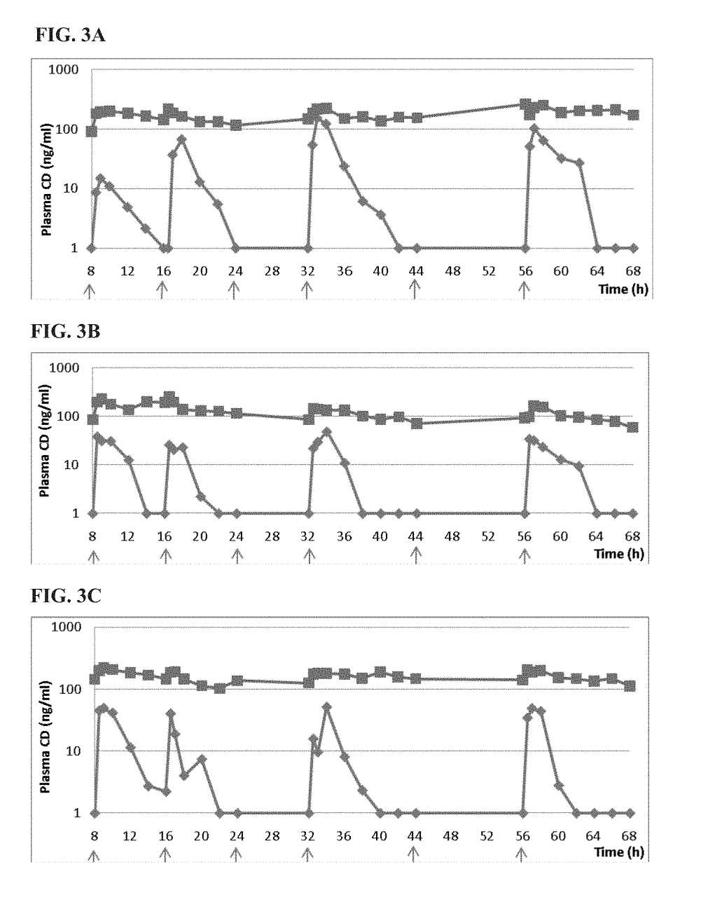

Results showed that the co-administration of continuous subcutaneous carbidopa with any oral LD preparation significantly increases (more than ×2) the half live (t½) and AUC of levodopa. In contrast, increased carbidopa oral dose or frequency did not considerably improve the PK profile of levodopa, as shown in Table 3. Also, constant, steady-state, levels of CD was maintained at 164±34 ng/ml during the 68 hours of continuous SC administration of carbidopa (60 mg/pig/day). This was in opposition to the fluctuating pattern and very low trough levels of CD obtained after administration of standard treatment (FIGS. 3A-3C). No signs of treatment related local or systemic toxicity were observed throughout the entire 68 h study period.

The pharmacokinetic parameters of levodopa determined in plasma of female landrace×large white swine (30-35 kg) following oral administration of (A) Stalevo (100/25/200 mg, LD/CD/E), (B) Dopicar+Lodosyn (125/25 mg LD/CD), (C) Sinemet CR (100/25 mg, LD/CD) Q8 and 12h, with or without continuous subcutaneous (SC) administration of 3% carbidopa (CD) solution, with results depicts in Table 3:

TABLE 3
A

| | Oral Treatment | | | | |
|---|---|---|---|---|---|
| | Stalevo (100/25/200 mg) | | | | |
| | PK Parameters | | | | |
| SC Treatment | Cₘₐₓ | Tₘₐₓ | T_{1/2} | AUC₀₋₈ | AUC_{0-∞} |
| Without SC CD | 2392 ± 1363.9 | 2.3 ± 0.89 | 1.4 ± 0.30 | 8109 ± 4145.2 | 8309 ± 4265.2 |
| (n = 8) | | | | | |
| With SC CD | 2355 ± 1157.1 | 2.1 ± 1.00 | 2.9 ± 0.41 | 17527 ± 8470.8 | 19330 ± 8284.8 |
| (n = 12) | | | | | |
| Significance* (p) | NS | NS | 2E−08 | 0.005 | 0.001 |

B

| | Oral Treatment | | | | |
|---|---|---|---|---|---|
| | LD/CD (125/25 mg) | | | | |
| | PK Parameters | | | | |
| SC Treatment | Cₘₐₓ | Tₘₐₓ | T_{1/2} | AUC₀₋₈ | AUC_{0-∞} |
| Without SC | 2472 ± 735.6 | 0.9 ± 0.53 | 1.1 ± 0.22 | 7200 ± 3093.2 | 7302 ± 3071.3 |
| CD(n = 7) | | | | | |
| With SC CD | 4050 ± 1369.5 | 0.8 ± 0.43 | 2.5 ± 0.43 | 17922 ± 4375.7 | 19230 ± 4625.5 |
| (n = 14) | | | | | |
| Significance* (p) | 0.005 | NS | 1E−07 | 7.4E−06 | 3.3E−06 |

C

| | Oral Treatment | | | | |
|---|---|---|---|---|---|
| | Sinemet CR (100/25 mg) | | | | |
| | PK Parameters | | | | |
| SC Treatment | Cₘₐₓ | Tₘₐₓ | T_{1/2} | AUC₀₋₈ | AUC_{0-∞} |
| Without SC CD | 1691 ± 556.2 | 0.9 ± 0.52 | 1.2 ± 0.19 | 4792 ± 1190.8 | 4929 ± 1196.6 |
| (n = 8) | | | | | |
| With SC CD | 2830 ± 929.2 | 1.2 ± 0.92 | 2.6 ± 0.46 | 12688 ± 3516.3 | 13505 ± 3344.4 |
| (n = 15) | | | | | |
| Significance* (p) | 0.002 | NS | 3.2E−08 | 2.3E−06 | 3.6E−07 |

*Using one tailed distribution equal variance T-Test

### Example 10

### The Effect of Continuous Subcutaneous Administration of Carbidopa on Brain Distribution of Levodopa and Dopamine in Mice

In this experiment, the purpose was to determine the effect of continuous subcutaneous administration of carbidopa (15 mg/kg/d) on the levels of levodopa and dopamine in the brain following oral administration of levodopa/carbidopa (32/8 mg/kg TID) in mice.

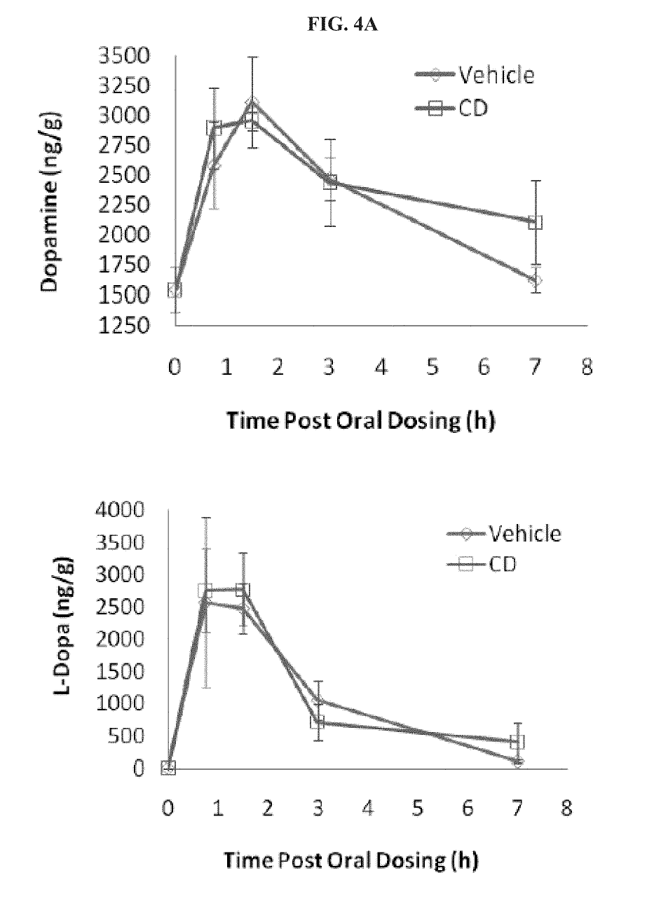

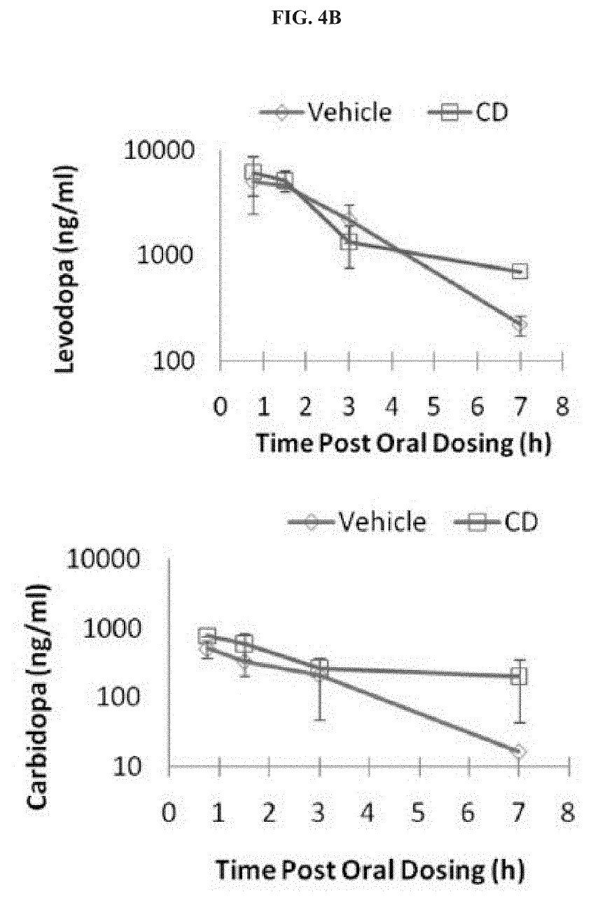

Mice were implanted subcutaneously with Alzet pumps containing Saline (Negative Control), Vehicle or Carbidopa solution. A day following implantation LD/CD was administered orally Q8h. The level of levodopa and dopamine in the brain was determined following the 4^{th} oral dose of LD/CD. The results showed dopamine levels seven hours post-administration of oral LD to be significantly higher in the brains of mice continuously administered SC with carbidopa, concurrently with higher levels of plasma LD (FIGS. 4A and 4B).

### Example 11

### Dose Effect of Continuous Subcutaneous Carbidopa Administration on Local Toxicity and Pharmacokinetic Profile of Levodopa and Carbidopa in Pigs

In this experiment, the purpose was to determine the dose effect of carbidopa continuously administered subcutaneously to pigs on local tolerance and the pharmacokinetics of L-dopa.

Pigs weighing 30-35 kg were administered orally with Sinemet® (Merck & Co., 100/25 mg, LD/CD,), thrice daily (q8h), or Dopicar® (Teva) Lodosyn® (Merck & Co., (125/25 mg LD/CD), twice daily (q12h), with continuous subcutaneous vehicle, 2% or 4% carbidopa (0, 40 or 80 mg/pig/d, respectively) for a total period of 24 h. Blood samples were collected at pre-determined time points and plasma levels of L-dopa and carbidopa were analyzed by LC-MS. Skin biopsies were collected from the infusion sites immediately, 1 and 2 weeks post-administration and local tolerance was evaluated by histological analysis of H&E stained slides. No histological treatment-related abnormalities were observed at the sites of infusion.

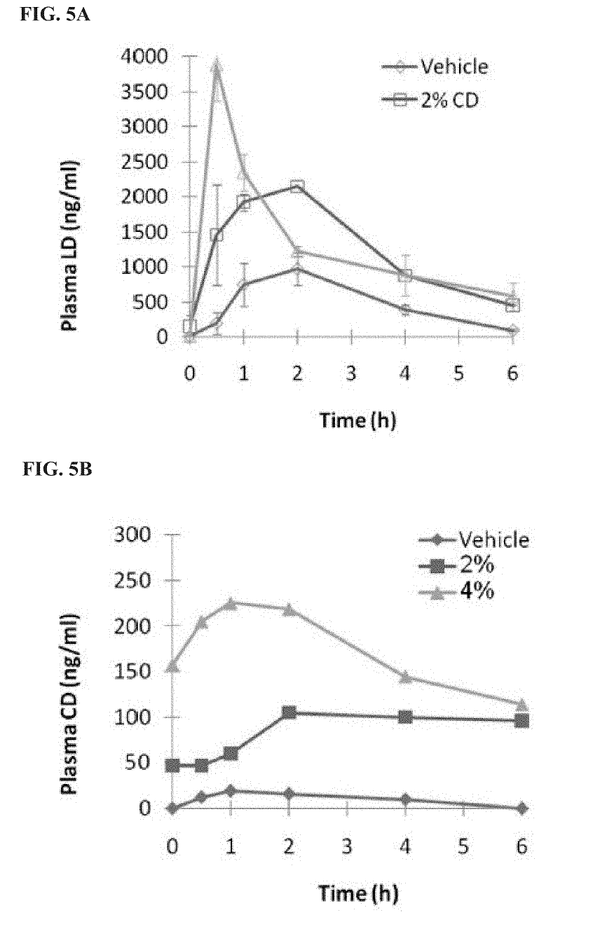

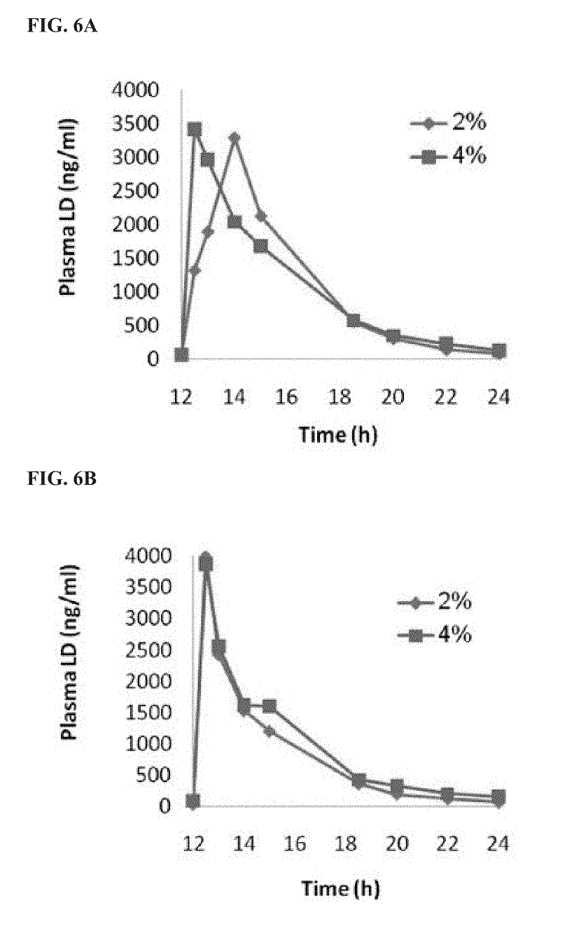

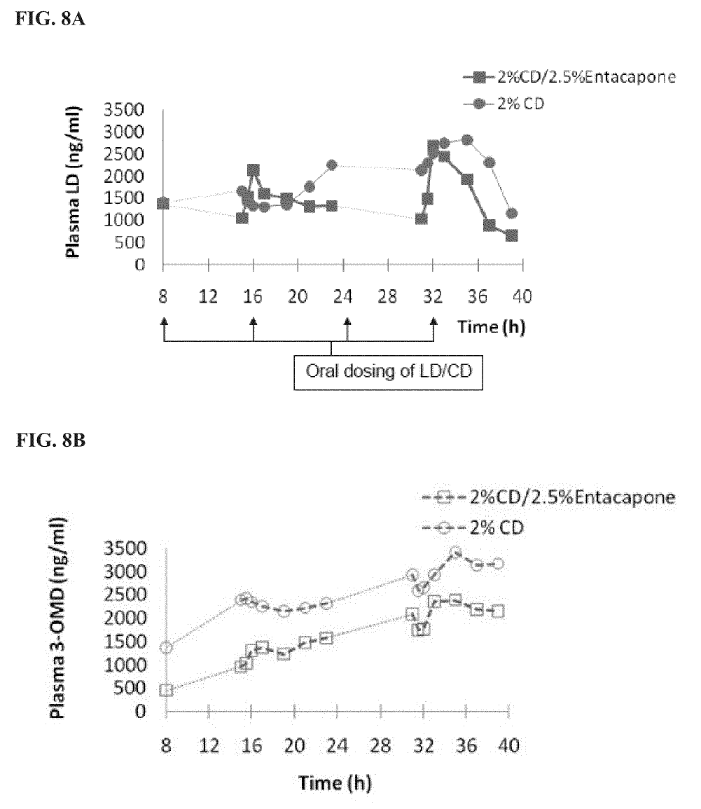

No significant dose effect on the plasma levels of L-dopa was observed when 2 or 4% carbidopa solutions were co-administered with Sinemet® (FIGS. 5A and 5B) or Dopicar®+Lodosyn® (FIGS. 6A and 6B). Thus, under the experimental conditions employed, it was suggested that continuous subcutaneous administration of 2% carbidopa, or less, may be sufficient to maintain optimal inhibition of DDC in pigs (see FIGS. 8A and 8B)

### Example 12

### The Effect of Continuous Subcutaneous Administration of Carbidopa, with and without Continuous Subcutaneous Administration of Entacapone, on the Pharmacokinetics of Levodopa in Pigs

In this experiment, the purpose was to determine the plasma levels of L-dopa, following continuous subcutaneous administration of carbidopa, with or without entacapone, concomitantly with oral administration of L-dopa/carbidopa in Pigs. Plasma levels of L-dopa were measured by HPLC-ECD. The results showed that entacapone effectively reduced the levels of 3-OMD, but it did not further extend the pharmacokinetics of levodopa, suggesting that entacapone and/or COMT inhibition interferes with carbidopa/DDC-dependent, or other, LD metabolic pathways, as shown in FIGS. 8A and 8B.

### Example 13

### The Effect of Continuous Subcutaneous Administration of Benserazide on the Pharmacokinetics of Levodopa in Pigs

In this experiment, the purpose was to determine the plasma levels of L-dopa, following co-administration of oral L-dopa/Carbidopa with continuous subcutaneous administration of another DDC inhibitor, benserazide. Plasma levels of L-dopa were measured by HPLC-ECD.

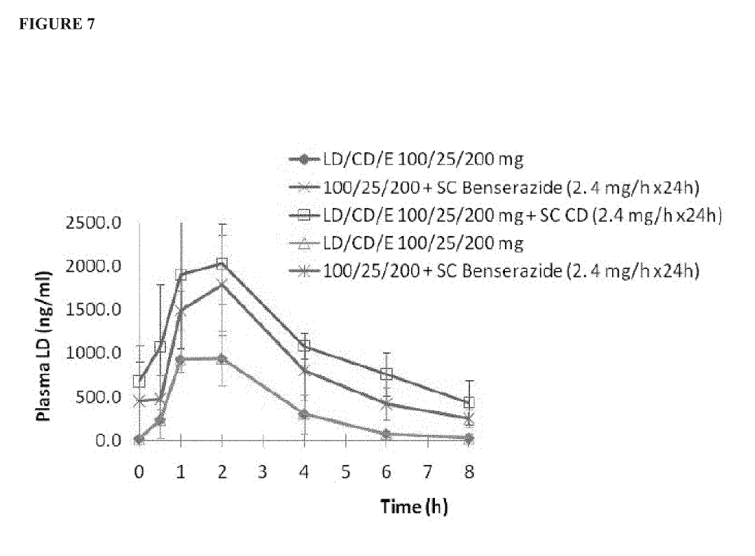

The results showed that benserazide extended the pharmacokinetic profile of LD, suggesting that continuous dopa-decarboxylase (DDC) inhibition, by any DDC inhibitor, increases the elimination half life of LD, as shown in FIG. 7.

### Example 14

### The Transdermal Delivery of Carbidopa Propyl Ester (CDPE) Through Full Thickness Pig Skin Ex Vivo using the Franz Cell Delivery System

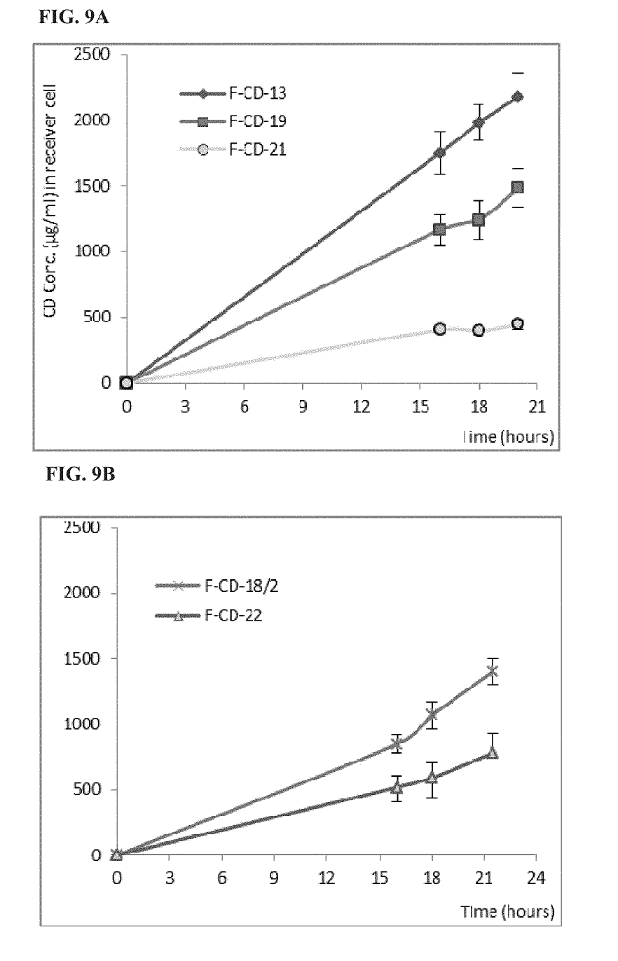

In this experiment, the purpose was to determine the transdermal delivery of carbidopa propyl ester through a full thickness porcine skin, ex vivo using the Franz cell delivery system. Gel formulations containing CDPE were prepared. Samples were collected from the receiver cell at time 0, 16, 19 and 22 hours after formulation application on to the skin. The amount of CD compounds in the receiver cell fluid was determined by a spectrophotometer at 280 nM. The results shown in FIGS. 9A and 9B demonstrate that CDPE penetrates the skin in an enhancer-dose dependent manner.

### Example 15

### The Effect of Oral Administration of Levodopa Arginine and Carbidopa Arginine Salts on the Pharmacokinetic Profile of Levodopa and Carbidopa

In this experiment, the purpose was to determine the pharmacokinetics of LD and CD administered orally as arginine salts, either enteric-coated or not. Pigs were orally administered with 255/45 mg LD-arginine salt (LDs)/CD-arginine salt (CDs) to 30-35 kg pigs in gelatin coated or non-coated capsules (corresponding to 100/25 LD/CD). Plasma levels of LD and CD were measured by HPLC-ECD.

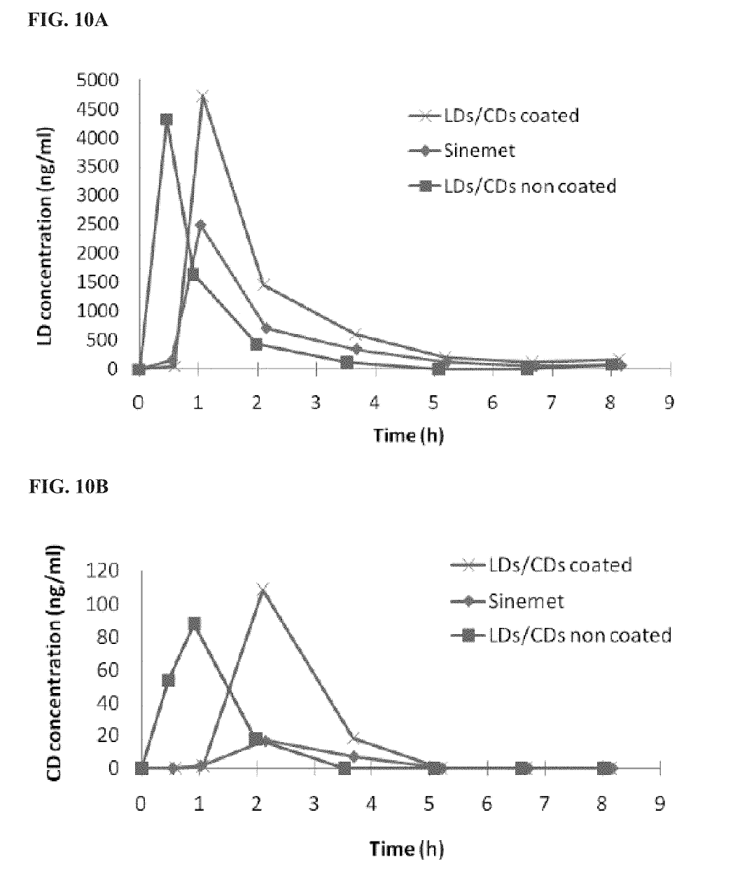

The results showed that LDs and CDs were absorbed more rapidly and efficiently as compared to LD/CD (Sinemet®), and that oral administration of enteric coated LDs/CDs extended the PK of plasma LD and CD (FIGS. 110A and 10B).

### Example 16

### The Inhibitory Effect of Carbidopa Esters on the Activity of Dopa-Decarboxylases (DDC) In Vitro

In this experiment, the purpose was to determine the inhibitory effect of carbidopa esters (CDEs) on the activity of dopa-decarboxylases. DDC enzymes were obtained from porcine liver homogenate and their activity was measured by comparing LD concentrations with and without carbidopa propyl ester (CDPE). Liver homogenate preparation was based on the method described by Umezawa. et al; (J. Antib. 1975, 28(12):947-52).

All samples were separated on high pressure liquid chromatography columns and the identity and concentration of L-DOPA and dopamine were determined by HP UV-HPLC analysis at 280 nM.

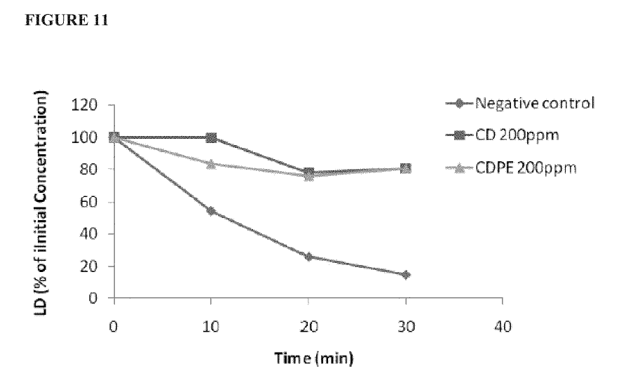

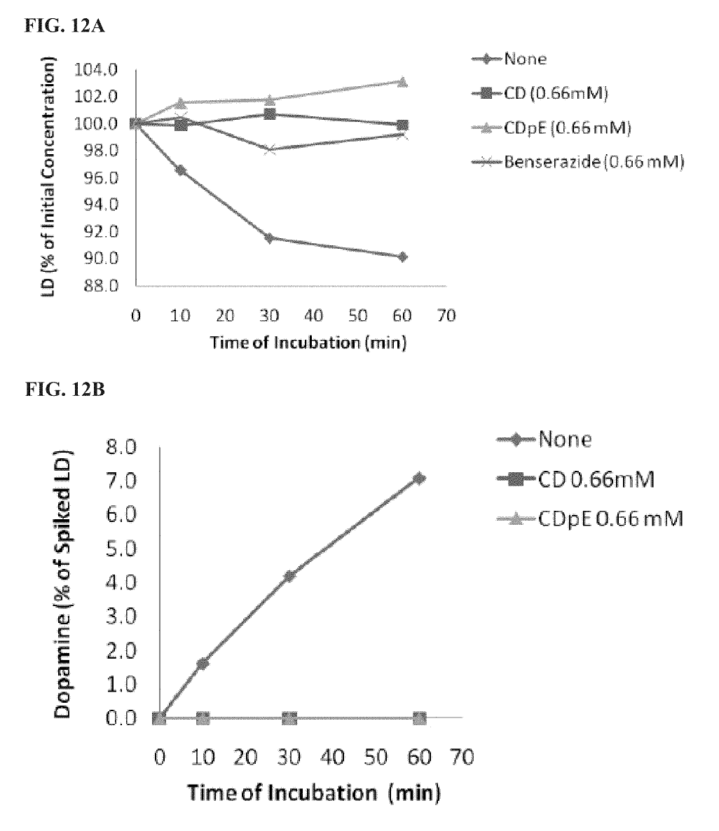

The results shown in FIGS. 11, 12A, and 12B demonstrate that CDpE inhibits the decarboxylation of L-dopa to dopamine, in a similar manner to carbidopa and benserazide.

### Example 17

### Synthesis and characterization of L-dopa and Carbidopa Alkyl Esters

Materials: L-β-3,4-dihydroxyphenylalanine (L-dopa, LD) and Carbidopa (CD) were purchased from Teva; Dry HCl (gas) was purchase from Maxima, butylated hydroxytoluene (BHT) from Sigma L-ascorbic acid 99%, from Aldrich; Sodium bisulfite from Merck; the alcohols for the synthesis of LD or CD alkyl esters, i.e., propanol, isopropanol, 1-hexanol, 1-octanol, butanol, triethyleneglycol methyl ether (TEGM), ethoxyethanol, propanediol were obtained from Sigma-Aldrich. Benzyl alcohol was purchased from Mallinckroft Chemicals, sodium hydrogen carbonate, ethyl acetate, and other compounds were purchased from commercial sources.

¹NMR was used in order to confirm the structure of the synthesis products. Analysis was done by the NMR services, Ben-Gurion University of the Negev (BGU), Casali Institute Hebrew University of Jerusalem, or Bar-Ilan University Israel. Mass spectroscopy (MS) was performed by the analytical services at Ben-Gurion University of the Negev (BGU), Israel.

For Thin Layer Chromatography (TLC), silica gel plates (Merck, OB 568397) were used for separating the products from the starting material using 89.5% Dichloromethane/10% Methanol/0.5% Acetic acid as the mobile phase

### HPLC-UV—Assay and Purity Profile for L-Dopa and Carbidopa Esters

HPLC system is used consisting of: Pump system; Diode Array Detector; Autosampler; Degasser; Column: Synergi, Fusion-RP, 80A 250×4.6 mm, 4 μ, under the following conditions: Wavelength: 280 nm; Flow rate: 1.3 ml/min; Injection volume: 40 μL; Stop time: 40 min, with solvents A Acetonitrile and B—20 mM Potassium Phosphate monobasic, pH=2.5 by H₃PO₄.

### Gradient

| Time | Solvent A | Solvent B |
|---|---|---|
| 0 | 2 | 98 |
| 5 | 2 | 98 |
| 15 | 60 | 40 |
| 20 | 60 | 40 |
| 25 | 2 | 98 |
| 30 | 2 | 98 |

Sample preparation: The tested ester was dissolved in a 100 mM HCl solution. Methanol is then added to obtain a 1:1 (v/v) HCl/methanol solution.

### Synthesis of LD Alkyl Esters with Thionyl Chloride

As an example, the synthesis of levodopa propyl ester is described as follows: n-Propanol (dried over CaO), 1800 ml (24 M), was placed into 5 L 3-necked round bottom flask (equipped with overhead stirrer, dropping funnel and condenser) and cooled to 0-(−2)° C. with Water:Ice:NaCl. Thionyl Chloride, 300 ml (4.1 M), was added drop wise to n-propanol at 0-4° C. (2.5-3 hours). L-DOPA, 300 g (1.52 M), was added in small portions with stirring and cooling. The stirring continued until full dissolution of L-DOPA.

The temperature was raised to 60° C. and stirred with heating overnight (approximately 16 h). The mixture was cooled to 40° C. and evaporated to dryness under reduced pressure. The residue (viscous mass) was cooled to room temperature, and then dissolved in 700 ml water. Sodium bicarbonate, 120 g (1.43 M), 160 g sodium sulfate and 0.3 g ascorbic acid were dissolved in 1.8 L deionized water, and the resulting solution was added to the reaction mixture. The pH of the solution was adjusted from pH 7 to approximately pH 8.

The precipitation of L-DOPA propyl ester started in the flask. The flask was left in the refrigerator for about 2 h. The product was filtered and, washed with cold water, containing 200 ppm ascorbic acid. The crude product was dried in vacuum oven at 30° C. ON and then 6 h at 50° C. The yield was 320 g crude product.

The water fraction was extracted twice with ethyl acetate (500 & 120 ml) containing 0.05% BHT. The organic fraction was separated and crystallized to afford 15 g product obtained with purity 99.2% (HPLC).

The crude product, 320 g, was dissolved in 3 L ethyl acetate containing 0.05% BHT at 68° C. The solution was filtered, cooled and left in the refrigerator for crystallization.

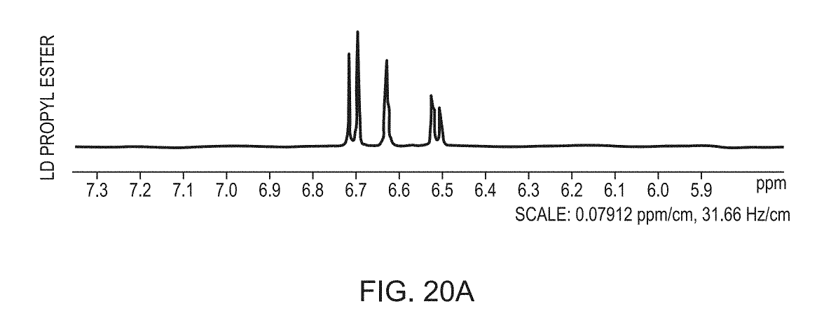

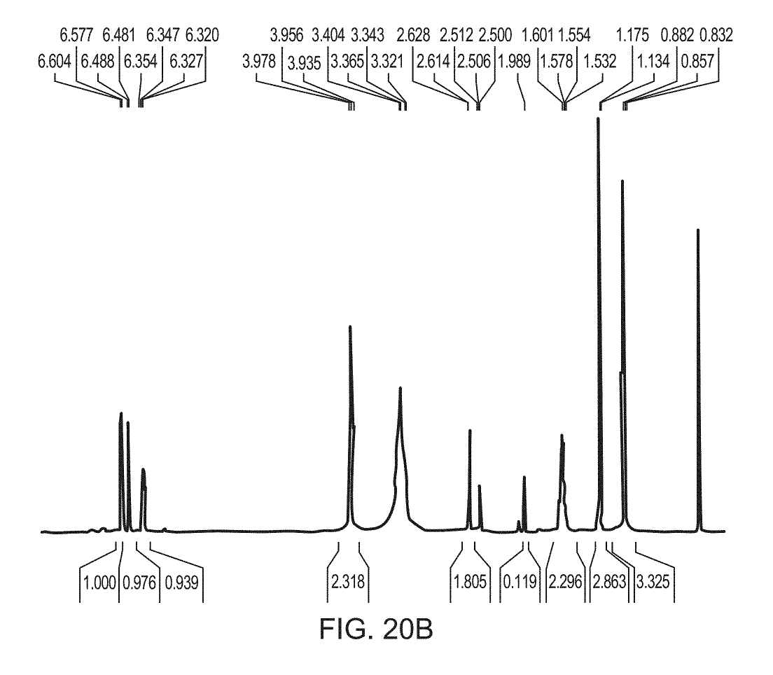

The crystalline material was filtered, washed with fresh ethyl acetate (containing BHT) and dried in vacuum oven overnight at 35° C. The yield of the crystallized product was 224 g with purity of 99.5% (HPLC). The NMR of the ester is shown in FIGS. 20A and 20B.

The combined ethyl acetate fractions were concentrated under reduced pressure to 750 ml, the residue was filtered and left for crystallization. An additional 40 g product was obtained with a purity of 99.3% (HPLC) and overall yield of 76.8%.

### Synthesis of LD Alkyl Esters with HCl Gas

As an example, the synthesis of levodopa octyl ester is described below:

To a round bottom 250 ml flask was placed 5 g (25.4 mmol) L-DOPA and 100 ml of 1-octanol. The mixture was cooled to 0° C. and kept under argon. HCl gas was introduced, and the mixture was heated to 130° C. for 2 h. The reaction was complete as determined by HPLC. The mixture was then cooled to 0° C. and transferred to 1 L Erlenmeyer flask.

Ethyl acetate (400 ml) and water (300 ml) were added to the reaction mixture and the pH was adjusted to 8 with sodium bicarbonate. The solution was then stirred for 30 minutes. The organic fraction was separated from water, washed with 300 ml brine and dried with anhydrous Sodium Sulfate.

After filtration, 10 mg BHT was added to the organic fraction, and the solution was concentrated to about 100 ml. n-Hexane (200 ml) was added to the stirred mixture.

A white crystalline product was obtained, and washed with hexane and diethyl ether and dried in the vacuum oven to constant weight. The yield was 5.4 g (63%). The product was pure as determined by TLC (1 spot at Rf 0.42) and HPLC. The structure was confirmed by NMR,.

### Synthesis of LD Benzyl Ester-HCl

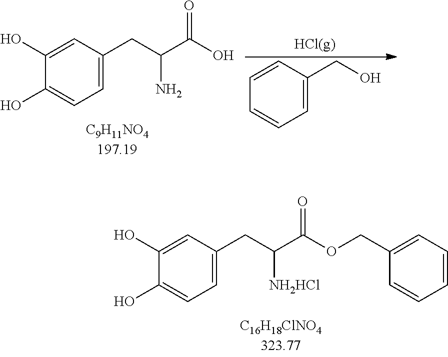

L-DOPA (20 grams, 101 mmoles) was mixed with benzyl alcohol (200 mL) and aqueous HCl (6N, 60 mL). The reaction mixture was stirred at 50° C. until the reaction mixture became a clear homogeneous solution. The reaction mixture was evaporated to remove water, in a rotoevaporator (using KNF diaphragm pump, 8-10 mm Hg). The water bath was maintained at 75° C. during the evaporation, until constant weight was obtained. The reaction mixture was cooled to room temperature, and HCl (gas) was introduced into the reaction mixture followed by stirring at 50° C. for 18 hours.

TLC: (Dichloromethane 89.5:10 Methanol:0.5 Acetic acid), Rf 0.34

The TLC plate was visualized with UV (short wave 254 nm) and by immersing the plate into KMNO₄ solution.

Evaporation of the water that was produced during the esterification was performed as described above, followed by addition of benzyl alcohol (100 mL) and introducing HCl (gas) for additional 8 minutes. The reaction mixture was stirred at 50° C. for additional 18 hours, The esterification reaction was monitored by TLC.

The reaction mixture was cooled to room temperature, dissolved in ethyl acetate (600 mL), stirred for 10 minutes. The white slurry was filtered and washed with ethyl acetate (2×200 mL). The white solid was re-dissolved in ethanol (200 mL) and stirred at 80° C., until complete dissolution.

The solution was then filtered, cooled to 50° C., and poured to ethyl acetate (650 mL) with stirring at room temperature. The white precipitate was filtered and washed with ethyl acetate (50 mL). The solid was dried in a vacuum oven (60° C., 18 hours). The product yield was 26.4 grams, 80.4%.

### Synthesis of LD Diol Esters

Propanediol-L-Dopa esters were prepared:

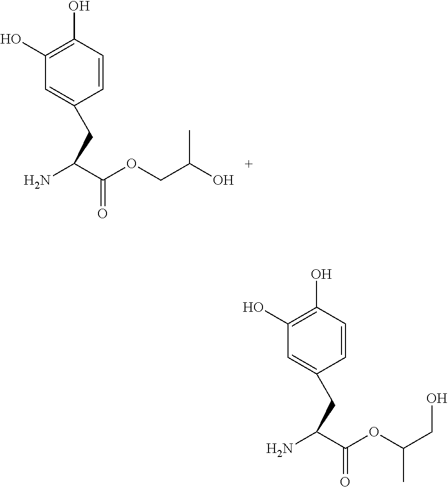

Structure was confirmed by H¹NMR analysis in DMSO. The assignments were δ8.8 (broad, 2H amine), 6.44 (2H, aromatics), 6.53 (1H, aromatics). 5.21(m, 1H), 3.82(m, 4H), 3.56 (1H, anomeric), 1.3 (3H, mixture of isomers).

Rf (methanol 1:9 dichloromethane) 0.3 and 0.44

### Synthesis of LD Octyl Ester

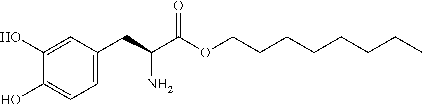 [SMILES: CCCCCCCCOC(=O)[C@@H](N)Cc1ccc(O)c(O)c1]

Structure was confirmed by H¹NMR analysis in CDCl₃. The assignments were δ6.72 (2H aromatics), 6.48 (1H, aromatics), 5.2 (2H, amine), 4.09 (2H, CH₂O), 3.83 (1H, anomeric), 2.8, 3.05 (2H, benzylic), 1.6 (2H, phenol) (12H, CH₂), 0.87 (3H, CH₃).

Rf (methanol 1:9 dichloromethane) 0.3 and 0.42

### Synthesis of CD Alkyl Esters

As an example, the synthesis of carbidopa ethyl and hexyl esters is described below:

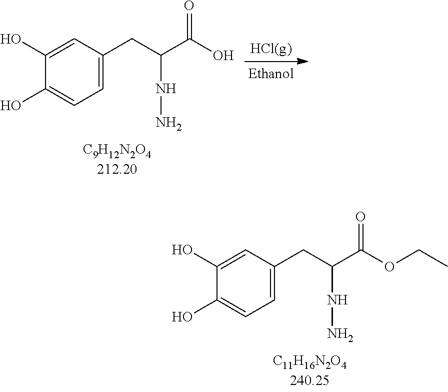 [SMILES: NNC(Cc1ccc(O)c(O)c1)C(=O)O ; CCOC(=O)C(Cc1ccc(O)c(O)c1)NN]

Carbidopa 20 g (0.088 M) was suspended in 400 ml Ethanol and dry HCl gas was introduced into the mixture. The mixture was stirred at 50° C. for 48 h. The solvent was evaporated at reduced pressure to dryness and the residue was treated with a mixture of 200 ml deionized water containing 5% sodium bicarbonate and 5% s metabisulfite.

The pH of the mixture was adjusted to pH 8 with a 5% sodium bicarbonate solution. A white product precipitated out of solution The product filtered, washed well with water, then ether and dried in the vacuum oven at 25° C. A white product (17.5 g) was obtained with yield of 77.3% which showed above 95% purity by HPLC.

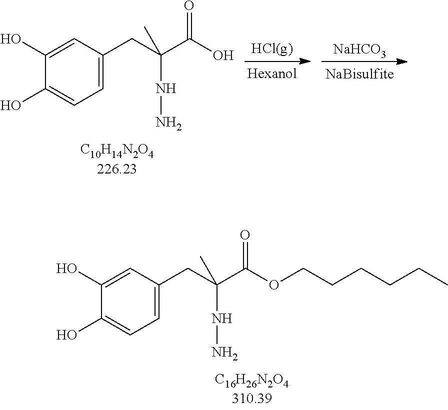 [SMILES: CC(Cc1ccc(O)c(O)c1)(NN)C(=O)O ; "NaHCO3" ; CCCCCCOC(=O)C(C)(Cc1ccc(O)c(O)c1)NN]

Carbidopa 20 g (0.088 M) was suspended in 300 ml hexanol. Dry HCl gas was introduced into the mixture and the mixture stirred at 85° C. for 12 h. The solvent was evaporated at reduced pressure to dryness and the residue was treated with a mixture of 200 ml deionized water containing 5% sodium bicarbonate and 5% sodium metabisulfite and ethyl acetate (300 ml, and BHT 50 mg).

The mixture was adjusted to pH-8 with 5% sodium bicarbonate solution. The organic layer was washed well with deionized water, then brine and dried over sodium sulfate anhydrous. The organic solvent was evaporated at reduced pressure to afford an oily product (21.2 gr, 95% purity by HPLC).

The carbidopa propyl ester was also prepared. The NMR of the ester is shown in FIG. 20B.

### Example 18

### Solubility in PBS

The “flask method” was used to determine the solubility of the test material in phosphate buffered saline (PBS) according to the OECD Guidelines for the Testing of Chemicals. Briefly, the test material was dissolved in PBS at a temperature slightly above the test temperature. When saturation is achieved, the solution is cooled to the test temperature and the mass concentration of the LD alkyl ester in the solution is determined quantitatively by measuring the absorbance of the solution at 280 nm and comparing the readings to a calibration curve of the relevant alkyl ester dissolved in PBS.

### Solubility of LDEs in PBS

| | LD-Ester (ug/ml) |
|---|---|
| Isopropyl | 17275 |
| Propyl | 11238 |
| Butyl | 8850 |
| Benzyl | 2250 |
| Octyl | 31 |

### Example 19

### Sensitization of LDEs (LLNA)

Sensitization was tested in mice using a LLNA (local lymph node assay) mod& for the detection of skin sensitization. Formulations containing 24% LD Propyl, Isopropyl or Ethyl esters were tested in DMF as compared to a Positive Control. In another LLNA study, LD Propyl and Benzyl Esters were tested in vehicle containing penetration enhancers.

The results in Tables 4A-B show that the Stimulation Index (SI) of LD Ethyl ester=Positive Control>>LDipE>LDpE when tested in DMF. However, the low SI of LDpE was significantly increased when applied using the transdermal formulation. In contrast, the SI of the LDbzE formulation was very low. The vehicle itself did not have a sensitizing effect (SI≦3). Based on the results, it may be suggested that various LD esters have different sensitizing potentials, and that LD Benzyl Ester has the lowest sensitizing potential as compared to other LDEs.

### Example 20

### Biodistribution and Metabolism of Levodopa Esters (LDEs) in Blood (Ex Vivo)

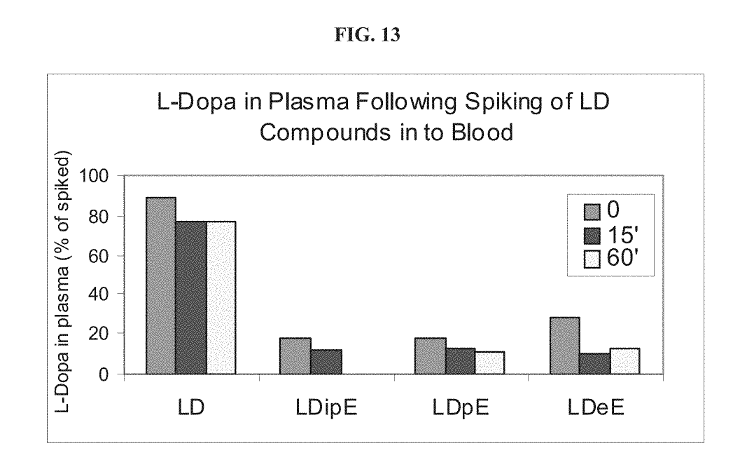

LDEs [isopropyl ester (LDipE), propyl ester (LDpE), ethyl ester (LDeE)] and LD-d₃, were spiked into whole blood, incubated for 15 or 60 minutes at 37° C., and the plasma or whole blood concentration of LD, LD-d₃ and LDipE were quantified using HPLC analysis as shown in Table 6 and FIG. 13.

TABLE 6

| Time | | | | | | | |
|---|---|---|---|---|---|---|---|
| after | | | | | | Total LD | |
| spiking | LDiPE | LD | LD-d₃ | LDiPE | LD | compounds | LD-d₃ |
| | LD in Blood (ng/ml) | | | Recovery from Blood (%) | | | |
| T₀ | 180 | 78 | 289 | 45 | 24 | 69 | 72 |
| T₁₅′ | 0 | 241 | 288 | 0 | 74 | 74 | 72 |
| | LD in Plasma (ng/ml) | | | Recovery from Plasma(%) | | | |
| T₀ | 0 | 37 | 533 | 0 | 5.7 | 5.7 | 67 |
| T₁₅′ | 0 | 41 | 573 | 0 | 6.3 | 6.3 | 72 |

Immediately after spiking, LDE was not found in plasma, but 45% was detected in whole blood. However, following incubation, LDE was not detected in plasma or whole blood. Recovery of LD compounds from plasma 15 minutes following spiking of LDipE and LD-d₃ into whole blood was 6% and 70%, respectively. In contrast, ±70% of the LD compounds was recovered from whole blood is following spiking of either LDipE or LD-d₃. These results appear to indicate that most of the spiked LDE rapidly enter red blood cells and LDE is rapidly hydrolyzed to LD, where the LD formed in the RBCs remain within the RBCs; LD does not enter the RBCs.

### Example 21

### Intradermal Delivery of LDE in Pigs

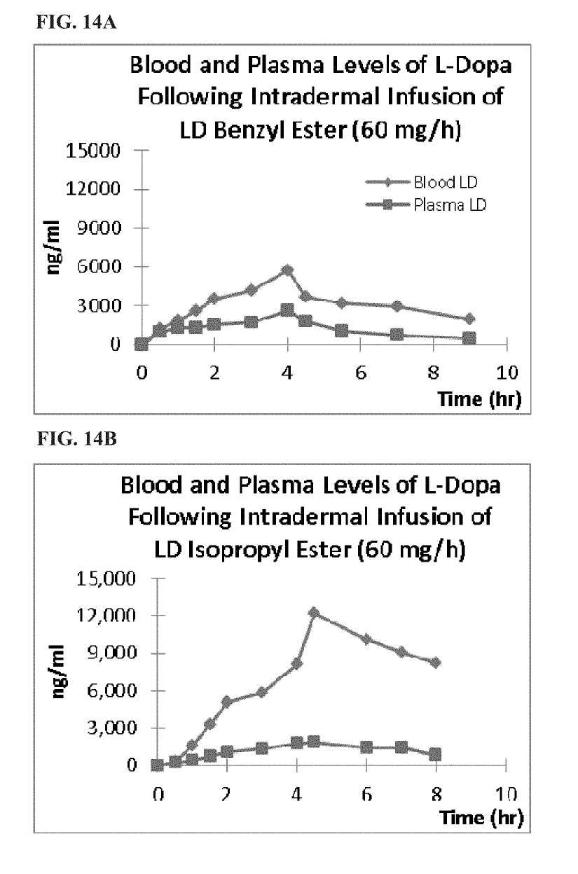

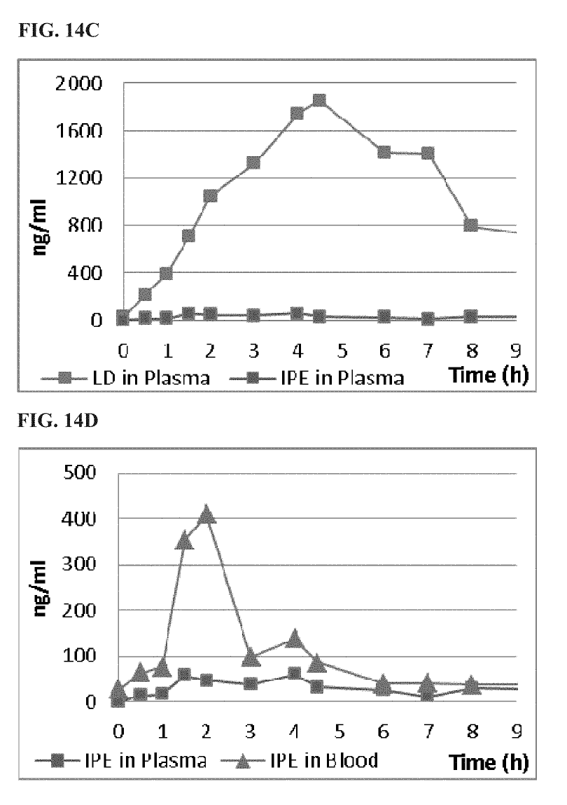

LDEs were infused intradermally for 4 hours. Blood samples were collected at pre-determined time points. Both whole blood and plasma samples were subjected to LD and LDE analysis. The analysis indicated that the levels of LD in plasma are significantly lower than in whole blood although steady state concentration of LD was not reached, suggesting that the levels obtained may increase upon extended continuous administration. Significant levels of LDipE were found in the RBC fraction during infusion, see FIGS. 14A-14D.

Some of the LDipE is hydrolyzed to LD in the plasma, but most of the LDipE appears to enter the RBCs and subsequently metabolize in the RBCs to LD. The latter is probably further metabolized in the RBCs. There seems to be no LD partitioning between the RBCs and the plasma. LD Benzyl Ester (LDbzE) appears hydrolyzed to LD faster than LD isopropyl ester. Therefore, more LD is found in the plasma, and respectively less LD is found in the RBCs, following LDbzE administration.

### Example 22

### Intravenous Infusion in Pig Models

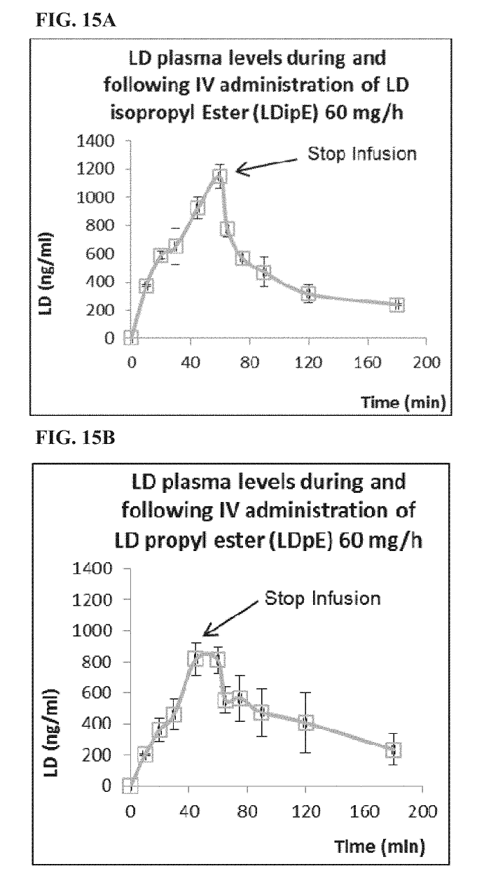

LD propyl ester (LDpE) and LDipE were infused continuously intravenously for 1h 60 mg/h in pig subjects. Blood samples were collected at pre-determined time points. Plasma was subjected to LD analysis. LD plasma levels of at least 1000 ng/ml were detected following 1 h continuous IV administration of LDE (60 mg/h), suggesting that at least some of the administered LDE was hydrolyzed to LD in the plasma. A steady state was not reached, suggesting that these levels may increase upon longer infusions. plasma levels are shown in FIGS. 15A and 15B.

### Example 23

### Pig Skin Penetration of LDE

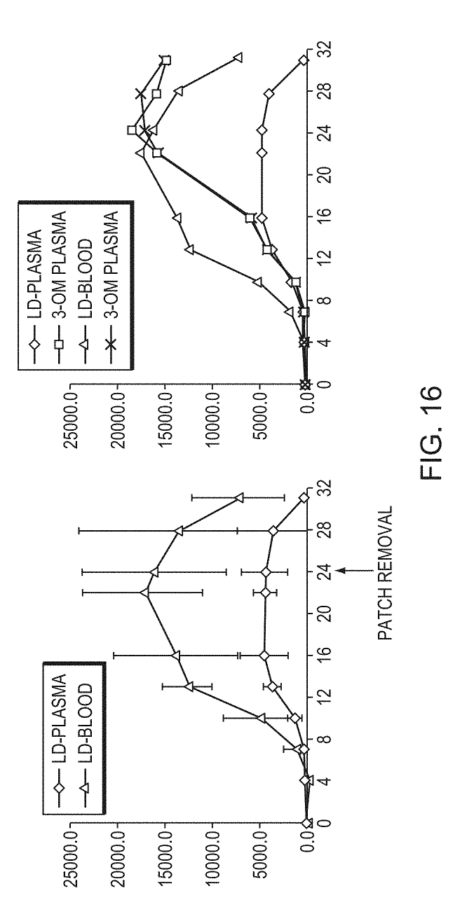

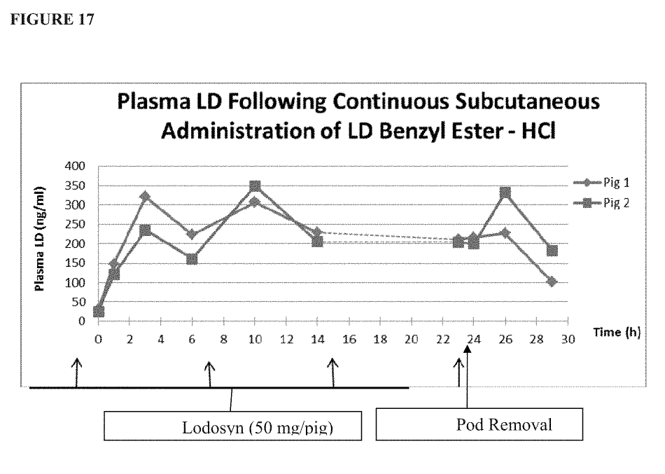

A LDE formulation was applied onto the back of pigs using two patches, each of 40 cm²/pig, as shown in FIG. 16. Patch removal occurred at 24 h. Blood samples were collected at pre-determined time points. Both whole blood and plasma were subjected to LD and 3-OMD analysis. Results indicate that LDE penetrates the skin following transdermal delivery and maintain steady state blood and plasma concentrations of LD, where most of the LDE rapidly enters the RBCs, where it is hydrolyzed to LD. LD formed in the RBC stay within the RBCs, and is further metabolized to 3-OMD, which is reallocated from the RBCs into the plasma. Table 7 depicts results:

Therapeutic plasma concentrations of LD are attained with all LDEs tested. The LD plasma concentration was 60% higher following LDbzE as compared to LDeE. The LD concentration in the blood cell fraction was ×2.5 and 1.9 higher following LDeE and LDpE administration as compared to LDbzE. The ratio between blood cell fraction LD and plasma LD was only ×1.6 following LDbzE administration as compared to ×3 and ×6.5 with LDpE and. LDeE respectively. Almost 40% of delivered LDbzE was found as LD in the plasma, whereas only 13 and 25% of the delivered ethyl and propyl esters, respectively, were found as LD in the plasma. The results suggest LDbzE may have less LD in blood cells and reduced amounts of the ester in the blood.

### Example 24

### Continuous Subcutaneous Administration of LDE in Pigs

An aqueous solution of 15% LD benzyl ester-HCl, pH 4.7, was continuously administered subcutaneously via an insulin patch pump, 2 ml/24 h. Carbidopa was administered orally q8h, starting 1 h prior to pump application. Blood samples were collected at pre-determined time points and the plasma concentrations of LD were analyzed. Results shown in FIG. 17. The results suggested that fluctuations of LD concentrations in the plasma coincided with oral carbidopa administration.

### Example 25

### Continuous Subcutaneous (SC) and Intradermal (ID) Administration of L-DOPA Ester (LDE) Formulations

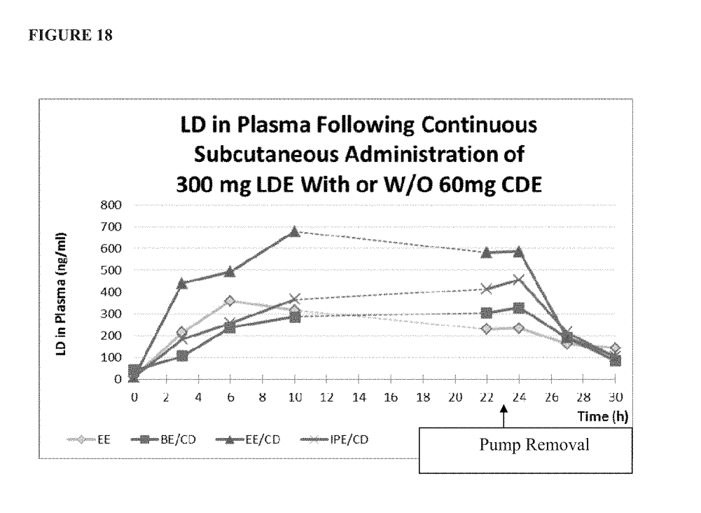

Formulations containing 1 5% LDE, with or without CDE (5:1), were continuously administered subcutaneously in pigs weighing 25-30 kg via insulin pumps for a period of 24 h, 0.08 ml/h. Blood was collected at pre-determined time points. Plasma and whole blood LD was quantified using HPLC, results shown in FIG. 18. The steady state (SS) plasma concentrations were attained following continuous SC administration of LDEs about 6 h after infusion initiation, thereafter, constant LD plasma concentrations were maintained. Continuous subcutaneous co-administration of CD ester increased the steady state plasma LD concentration by at least×2 (EE vs. EE/CD).

### Example 26

### Effect of pH on the Stability of LDipE

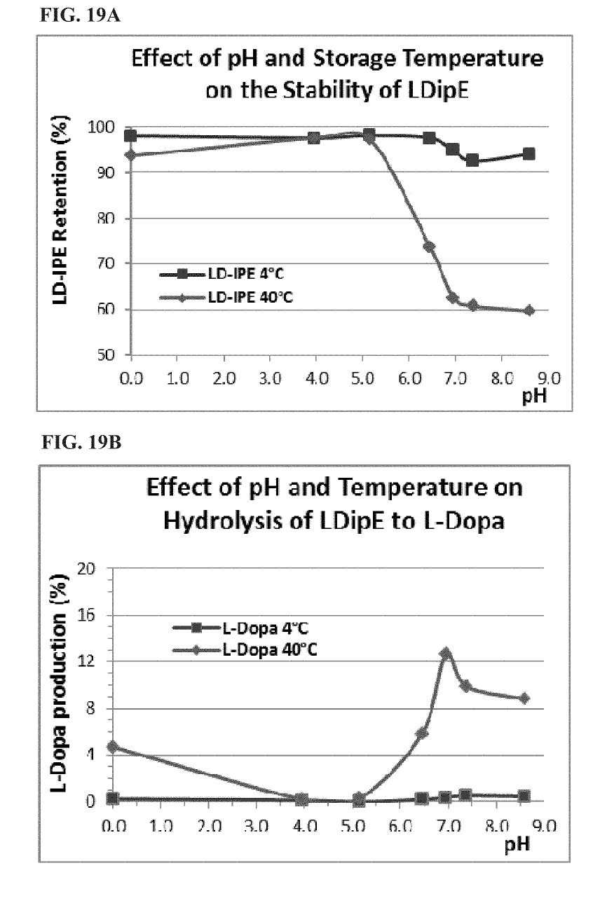

FIGS. 19A and 19B indicate the effect of pH on stability and hydrolysis of LDipE after 10 days at 4 and 40° C. At 4° C. the most stable formulation tested was at pH 5.1, and at 40° C. the most stable formulations tested was at pH 3.7 and 4.8. The results suggest that the LDipE formulation is stable at pH ranging between 3 and 6.5 for at least 24 h at 2-8° C.; and LDipE formulation is stable at pH ranging between 3 and 5.5 for at least 24 h at 40° C.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

### INCORPORATION BY REFERENCE

The entire contents of all patents, published patent applications, websites, and other references cited herein are hereby expressly incorporated herein in their entireties by reference.

1. A pharmaceutically acceptable liquid composition comprising:
a levodopa ester or pharmaceutically acceptable salt thereof, wherein the ester is selected from the group consisting of: levodopa methyl ester, levodopa ethyl ester, levodopa propyl ester, levodopa isopropyl ester, levodopa benzyl ester, levodopa butyl ester, levodopa octyl ester, levodopa triethyleneglycol methyl ether ester, levodopa propanediol ester, and levodopa ethoxyethyl ester; and
water
wherein the liquid composition has a pH of about 3.0 to about 9.5 at 25° C. and is stable at 25° C. for 48 hours.

2-4. (canceled)

5. The pharmaceutically acceptable liquid composition of claim 1, wherein the liquid composition has a pH of about 4.0 to about 6.0 at 25° C.

6. The pharmaceutically acceptable liquid composition of claim 1, wherein the liquid composition has a pH of about 6.0 to about 8.0 at 25° C.

7. The pharmaceutically acceptable liquid composition of claim 1, wherein the levodopa ester salt is an organic acid salt.

8. The pharmaceutically acceptable liquid composition of claim 1, wherein the pharmaceutically acceptable levodopa ester salt is selected from the group consisting of an HCl salt, a tartrate salt, a succinate salt, an arginine salt, a fumarate salt, an adipate salt, an aspartate salt, and a glutamate salt.

9. The pharmaceutically acceptable liquid composition of claim 1, comprising about 5% (w/v) to about 50% (w/v) levodopa ester or a pharmaceutically acceptable salt thereof.

10. The pharmaceutically acceptable liquid composition of claim 1, comprising about 10% (w/v) to about 99% (w/v) water.

11. The pharmaceutically acceptable liquid composition of claim 1, further comprising carbidopa or a pharmaceutically acceptable salt or ester thereof.

12. The pharmaceutically acceptable liquid composition of claim 11, wherein the pharmaceutically acceptable salt or ester of carbidopa is selected from the group consisting of arginine salt, methyl ester, ethyl ester, propyl ester, isopropyl ester, benzyl ester, butyl ester, octyl ester, triethyleneglycol methyl ether ester, propanediol ester, and ethoxyethyl ester.

13. The pharmaceutically acceptable liquid composition of claim 1, further comprising a pharmaceutically acceptable excipient.

14. The pharmaceutically acceptable liquid composition of claim 13, wherein the pharmaceutically acceptable excipient is selected from the group consisting of N-methylpyrrolidone, polyvinylpyrrolidone, propylene glycol, polyethylene glycol, antioxidants, and combinations thereof.

15.-22. (canceled)

23. A pharmaceutically acceptable liquid composition comprising:
a levodopa ester or a pharmaceutically acceptable salt thereof, wherein the a levodopa ester is selected from the group consisting of: levodopa methyl ester, levodopa ethyl ester, levodopa propyl ester, levodopa isopropyl ester, levodopa benzyl ester, levodopa butyl ester, levodopa octyl ester, levodopa triethyleneglycol methyl ether ester, levodopa propanediol ester, and levodopa ethoxyethyl ester,
carbidopa or a pharmaceutically acceptable salt or ester thereof, and
water;
wherein the composition is substantially stable at 25° C. for 48 hours or more.
